(19) United States
(12) Patent Application Publication
(10) Pub. No.: US 2002/0177172 A1
(43) Pub. Date: Nov. 28, 2002

(54) RESTORATION OF PLATELET AGGREGATION BY ANTIBODY ADMINISTRATION AFTER GPIIB/IIIA ANTAGONIST TREATMENT

(76) Inventors: KEVIN CHALLON GLENN, MARYLAND HEIGHTS, MO (US); CHRIS P. CARRON, WILDWOOD, MO (US); LARRY P. FEIGEN, WAUCONDA, IL (US); JIMMY D. PAGE, GURNEE, IL (US); JODI A. PEGG, BALLWIN, MO (US)

Correspondence Address: G D SEARLE & CO, ROGER A WILLIAMS, CORPORATE PATENT LAW DEPARTMENT, P O BOX 5110, CHICAGO, IL, 606805110

(21) Appl. No.: 09/195,133

(22) Filed: Nov. 18, 1998

Related U.S. Application Data
(63) Continuation-in-part of application No. 08/856,761, filed on May 15, 1997, now Pat. No. 6,150,114.
(60) Provisional application No. 60/017,710, filed on May 15, 1996.

(51) Int. Cl. G01N 33/53; G01N 33/567

(52) U.S. Cl. 435/7.2

(57) ABSTRACT

The invention provides a process to restore platelet aggregation by the administration of antibody combining site-containing molecules that specifically bind to a specific class of reversibly-bound GPIIb/IIIa fibrinogen receptor antagonist compounds.

### CROSS-REFERENCE TO RELATED APPLICATION

This is a continuation-in-part of U.S. application Ser. No. 08/856,761 filed May 15, 1997.

### TECHNICAL FIELD

This invention is directed to the restoration of platelet aggregation by the administration of antibody combining site-containing molecules that bind to the fibrinogen receptor antagonists, and more particularly to administering antibody combining site-containing molecules that bind to a specific class of reversibly-bound GPIIb/IIIa receptor antagonist compounds.

### BACKGROUND OF THE INVENTION

Fibrinogen is a glycoprotein present as a normal component of blood plasma. Fibrinogen participates in platelet aggregation and fibrin formation in the blood clotting mechanism.

Platelets are cellular elements present in whole blood that also participate in blood coagulation. Platelets have a beneficial function in the cessation of blood flow (hemostasis) by providing an initial hemostatic plug at sites of vascular injury.

Generally, the platelet first adheres to macromolecules in the subendothelial regions of an injured blood vessel and then platelet aggregates form the primary hemostatic plug. The aggregation of platelets near the injury activates plasma coagulation factors that lead to the formation of a fibrin clot that supports and reinforces the aggregate.

Measurement of activated clotting times (ACT) was developed by Hattersly as a sensitive test to monitor whole blood clotting. Hattersly, P. G., J. Am. Med. Assoc., (1966) Vol. 196, pp. 150-154. Others have used the test as an assay to demonstrate drug activity. Moliterno et al. describe the increase of activated clotting times when the anti-GPIIb/IIIa antibody C7E3 is administered. Moliterno, D. et al. Am. J. Cardiol., (1995) Vol. 75, pp. 559-562.

Fibrinogen binding to platelets is important to normal platelet function in the blood coagulation mechanism. When a blood vessel receives an injury, the platelets binding to fibrinogen initiate aggregation and form a thrombus. Interaction of fibrinogen with platelets occurs through a membrane glycoprotein complex, known as GPIIb/IIIa; this interaction is an important feature of the platelet function.

It is also known that another large glycoprotein named fibronectin, which is a major extracellular matrix protein, interacts with fibrinogen and fibrin, and with other structural molecules such as actin, collagen and proteoglycans. Several relatively large polypeptide fragments in the cell-binding domain of fibronectin have been found to exhibit cell-attachment activity.

The activation of platelets and resultant aggregation have been shown to be important factors in the pathogenesis of unstable angina pectoris, transient myocardial ischemia, acute myocardial infarction and atherosclerosis. In most of these serious cardiovascular disorders, intracoronary thrombus is present. The thrombus is generally formed by activated platelets that adhere and aggregate at the site of endothelial injury.

Thrombosis is a process in which a platelet aggregate and/or fibrin clot blocks a blood vessel. A thrombus blocking an artery can lead to the death of the tissue that is supplied blood by that artery. This blockage causes conditions such as stroke, unstable angina and myocardial infarction. Thrombosis can also cause complications after surgical procedures. For example, blood clots can form at sites that have been opened for implantation of prostheses, such as artificial heart valves, or for percutaneous transluminal angioplasty (PCTA).

Because of the relative contribution of activated platelets to aggregation and subsequent formation of an occulusive thrombus, antiplatelet agents have been developed that inhibit platelet aggregation. These agents are directed at the treatment and prevention of such complications arising from atherosclerosis and pathological thrombosis.

Many antiplatelet compounds having different functions are described in the art. Current antiplatelet agents include aspirin (ASA), which mainly interupts the thromboxane pathway; ticlopidine, which predominately interferes with the ability of adenosine diphosphate (ADP) to stimulate platelets; and thromboxane A₂ synthase inhibitors, which act against thromboxane A₂. Antiplatelet compounds like ASA act irreversibly, diminishing a treated platelet's ability to participate in a clotting event for the lifetime of the treated platelet.

Several patents disclosing antiplatelet compounds have issued and applications for patents disclosing additional compounds have been published. For example, U.S. Pat. No. 5,344,957 (Bovy et al.) discloses substituted β-amino acid derivatives useful as platelet aggregation inhibitors and PCT Application Publication No. WO 94/22820 (Abood et al., published Oct. 13, 1994) discloses 1-amidinophenyl-pyrrolidones piperidinones and azetinones useful as platelet inhibitors. The disclosures of that patent and published application, including the art cited therein, are hereby incorporated into this specification to more fully define the state of the art.

The new generation of antiplatelet agents called glycoprotein (GP) IIb/IIIa receptor antagonists function by reversibly disrupting the fibrinogen-platelet glycoprotein IIb/IIIa (“GPIIb/IIIa”) interaction and are active inhibitors of all platelet activating agents. Zablocki, J. A. et al., Exp. Opin. Invest. Drugs, (1994) Vol. 3(5), pp. 437-448; WO 97/35592; Reilly, T. M. et al., Ateriosclerosis, Thrombosis, and Vascular Biology, December, 1995, Vol. 15(12), p 2195-9).

These antagonists act by blocking fibrinogen (fgn) binding at the arginine-glycine-aspartate (RGD) recognition sequence on the GPIIb/IIIa receptor of activated platelets. The binding of fgn to the GPIIb/IIIa receptors is considered the final common pathway of platelet aggregation that leads to thrombus formation. These new agents effectively inhibit the formation of platelet aggregates, and consequently, their use provides an effective therapeutic process for modulating or preventing platelet thrombus formation.

Exemplary antagonists directed against the GPIIb/IIIa complex include antibody C7E3 (Centocor); compounds MK383: N-(butylsulfonyl)-0-(4-(4-piperidinyl)butyl)-L-tyrosine, monohydrochloride (Merck, West Point, Pa., USA 19486-0004); L-703014: (R)-beta[[[[1-oxo-4(4-piperidinyl)butyl]amino]-acetyl]amino]-1H-indole-3-pentanoic acid (Merck); RO 44-9883: (S)-[[1-[2-[[4-(aminoiminomethyl)benzoyl]-amino]-3-(4-hydroxyphenyl)-1-oxopropyl]-4-piperidinyl]oxy]acetic acid (HoffmanLaRoche Nutley, N.J., USA 07110-1199); GR 144.053: 4-(4-(4-(aminoiminomethyl)phenyl)-1-piperazinyl)-3-methyl-1-piperidine acetate (Glaxo, Research Triangle Park, N.C., U.S.A. 27709); BIBU 104: methyl trans-5-(S)-[[4-[4-(imino[(methoxycarbonyl)-amino]methyl]phenyl]-phenoxy]methyl]-2-oxopyrrolidine-3-acetate (Boehringer Ingleheim, Ridgefield, Conn., U.S.A. 06877-0368); cyclic peptide DMP 728: cyclic [D-2-aminobutyryl-N2-methyl-L-arginyl-glycyl-L-aspartyl-3-aminomethyl-benzoic acid] methanesulfonic acid salt; (DuPont Merck, Wilmington, Del., U.S.A. 19805) and the cyclic heptapeptide Integrelin™ (COR Therapeutics, So. San Francisco, Calif., USA).

However, administration of these new antiplatelet antagonists to inhibit platelet aggregation can lead to undesirable and severe hemorrhagic events. (Reilly, T. M. et al., Ateriosclerosis, Thrombosis, and Vascular Biology, December, 1995, Vol. 15(12), p 2195-9). Thus, a therapeutic process that restores the aggregation activity of platelets treated with a previously bound GPIIb/IIIa antagonist would be beneficial.

### BRIEF SUMMARY OF THE INVENTION

The disclosure that follows provides a process that utilizes antibody combining site-containing molecules that specifically bind to (immunoreact with) reversibly-bound GPIIb/IIIa receptor antagonist molecules and thereby restore the ability of platelets treated with such GPIIb/IIIa receptor antagonists to aggregate and form clots.

It has now been discovered that antibody combining site-containing molecules (collectively referred to as antibodies or in the singular as an antibody for ease of discussion) that immunoreact with a reversibly-bound GPIIb/IIIa receptor antagonist compound (GPIIb/IIIa antagonist) affect the activated clotting time (ACT) or platelet aggregation of blood containing that compound. Additionally, by administering a pharmaceutically effective amount of the antibody to a subject that has previously received such a GPIIb/IIIa receptor antagonist, platelet aggregation can be rapidly restored, resulting in restored hemostatic function in the subject.

Thus, this process begins with a mammalian host (human patient or other mammalian subject in need thereof) that has been treated with a reversibly-bound GPIIb/IIIa receptor antagonist compound that exhibits a plasma half-life of about two hours to about thirty-six hours, and a GPIIb/IIIa receptor off-rate of about 0.7/seconds (t1/2˜1 second) to about 0.012/seconds (t1/2˜60 seconds). The process comprises the steps of:

(a) contacting the blood of that host with a therapeutically effective amount of antibody combining site-containing molecules that specifically bind to the GPIIb/IIIa receptor antagonist compound to form antibody-treated blood. In the second process step, (b), the antibody-treated blood is maintained for a period of time sufficient to restore platelet aggregation.

The contemplated antibody combining site-containing molecules specifically bind to a reversibly-bound GPIIb/IIIa receptor antagonist compound that exhibits a plasma half-life of two hours to thirty-six hours and a GPIIb/IIIa receptor off-rate of about 0.7/seconds (t1/2˜1 second) to about 0.012/seconds (t1/2˜60 seconds). More preferably, the plasma half-life is about 6 hours to about 18 hours, and the GPIIb/IIIa receptor off-rate is about 0.2/seconds (t1/2˜3 seconds)to about 0.02/seconds (t1/2˜30 seconds). The antibodies described herein specifically bind to and inhibit the pharmacological activity of the GPIIb/IIIa antagonists. Intact antibodies can be used as can molecules that are free of immunoglobulin Fc portions or are single chain Fv proteins produced by recombinant methods or phage display of H and L chain variable domains. Those antibody combining site-containing molecules can be monoclonal or polyclonal and can be monovalent, divalent, up to decavalent. Two preferred GPIIb/IIIa receptor antagonist compounds with which the mammalian hosts are treated are compound B and compound D whose names and structures are disclosed hereinafter, or a pharmaceutically acceptable salt thereof.

The present invention has several benefits and advantages. One benefit is that following administration of a GPIIb/IIIa antagonist to a host, administration of antibody combining site-containing molecules that specifically bind to (immunoreact with) a reversibly-bound GPIIb/IIIa antagonist can ameliorate hemorrhagic complications related to such administration by restoring primary hemostatic function in a host.

An advantage of the invention is that its use and the subsequent restoration of primary hemostatic function in a subject would be advantageous to the subject by reducing blood loss that can occur if the pharmacological activity of the GPIIb/IIIa antagonist were not ameliorated.

Additionally, a subject treated with a GPIIb/IIIa antagonist can require emergency surgical intervention in which case restoration of primary hemostatic function to levels near pre-GPIIb/IIIa antagonist administration levels should be achieved before such intervention can safely be performed. The present invention beneficially provides a method of ameliorating the pharmacological activity of the GPIIb/IIIa antagonist and restoring primary hemostatic function and reducing the risk of severe hemorrhagic events during surgery in a subject previously treated with a GPIIb/IIIa antagonist.

A GPIIb/IIIa antagonist compound is contemplated for use prophylactically to prevent thrombotic complications associated with atherosclerosis or other coronary heart disease. The use of GPIIb/IIIa antagonists as a prophylactic can create a situation whereby administration of the GPIIb/IIIa antagonist can occur outside the controlled environment of a hospital or other medical facility where emergency treatment (e.g., hemodialysis or platelet transfusion) is readily available to reduce possible bleeding complications. In such a situation a hemorrhagic injury sustained by the host can be life threatening and extraordinary steps must be taken to limit the loss of blood. Thus it is advantageous to have an agent that is available in an easy to administer “out-patient” formulation. It is contemplated that up to 2 percent of such hosts taking a GPIIb/IIIa antagonist as a prophylatic will require such emergency ambulatory treatment in which platelet function must be restored to near normal levels. The present invention contemplates a method for treating such patients.

Still further benefits and advantages of the invention will be apparent to the skilled worker from the disclosure that follows.

### BRIEF DESCRIPTION OF THE DRAWINGS

In the drawings forming a portion of this disclosure:

FIG. 1 is a graph that shows the effect on the measured activated clotting time (ACT) for various concentrations of two antiplatelet compounds: compounds B (black square) and D (black dot). The structures of these compounds are given in the Detailed Description of the Invention. Concentrations are represented as multiples of the IC₅₀ values for each compound. The IC₅₀ value of compound B is 27 ng/mL and the IC₅₀ value of compound D is 43 ng/mL;

FIG. 2 is a bar graph that shows a comparison of activated clotting time ratios for blood samples with and without antibodies;

FIG. 3 is a bar graph that illustrates the effects of several enumerated monoclonal antibodies (“Mabs”) at a concentration of 60 nM in a platelet aggregation assay for their ability to recognize and neutralize antiplatelet compounds B, D and the Merck compound MK383 which was used as a control for antibody specificity. The levels of antiplatelet compounds used gave at least 50% inhibition of aggregation by themselves;

FIG. 4 is a bar graph that illustrates a comparison of activated clotting times in seconds (sec) for blood containing the antiplatelet compound B alone (B), as well as in the presence of enumerated monoclonal antibodies. “Control” represents the clotting time of the blood with no compound B and no anticompound B antibody present. “Cont Mab” is the clotting time for an antibody directed against an irrelevant protein;

FIG. 5 is a bar graph showing the clotting time in seconds (sec) for whole blood from two different donors (6 and 7) in the absence of any antagonist compound (open bars), in the presence of antiplatelet compounds B and D present at 250 nM (Controls) and in the presence of one or the other of those compounds plus 400 nM of monoclonal antibody 9F7;

FIG. 6 is a bar graph showing the restoration of platelet aggregation function by the monoclonal antibodies 9F7 and a series of crude goat (i.e. not affinity-purified) polyclonal IgG preparations in the presence of either 50 nM compound B (black bars) or 100 nM compound D (gray bars);

FIG. 7 is a bar graph that illustrates the reversal of human platelet aggregation inhibition (mean +/−SEM) in vitro by Mab 9F7 after platelets were incubated for 2 minutes with either of two concentrations of compound B (black bars=50 nM; gray bars=100 nM) in the presence or absence of monoclonal antibody 9F7 present at about a 60 nM concentration, with aggregation being induced by the addition of 4 μg/mL collagen and the extent of aggregation being measured after 3 minutes;

FIG. 8 is a graph showing the in vivo reversal of i.v.-administered compound B inhibition of platelet aggregation by Mab 9F7 in guinea pigs after an i.v. infusion of compound B was administered to reach steady state and then terminated (Stopped 2 Hr Infusion). A saline solution was infused in the control animals (diamonds) and platelet aggregation was monitored for 4 hours after the termination of the drug infusion, whereas the treated animals (squares) were immediately started on an infusion of 1.67 mg/min Mab 9F7 for 60 minutes and aggregation followed for 4 hours;

FIG. 9 is similar to that of FIG. 8, but showing in vivo reversal of platelet inhibition in dogs by Mab 9F7 after an i.v. infusion of compound B. The average recovery of platelet aggregation (±SEM) of three control dogs (circles) is shown following the termination of infusion of compound B. A dog that received the same infusion was subsequently infused with 9F7 at 1.67 mg/minute for 60 minutes and the aggregation function determined, with data being shown as the recovery of aggregation function (squares);

FIG. 10 is a graph that shows the effect of three bolus doses of about 50 mg of Mab 9F7 on the percent inhibition of platelet aggregation (ovals) and free compound B (rectangles) in vivo in a dog treated orally with 10 mg of compound A BID for the prior four days followed by anesthetization, in which B1, B2 and B3 represent the first, second and third bolus injections, respectively, and the numbers thereafter indicate the time in minutes after each bolus that the blood samples were taken. The free compound B is that compound not bound to antibody and thus able to bind to the platelet fibrinogen receptor and inhibit platelet aggregation;

FIG. 11 is a graph showing the total amount of compound B (squares) and the amount of free compound B (ovals) from the study of FIG. 10. The total amount of compound B is both free and antibody-bound compound and illustrates that the total amount of compound increases over the course of the experiment due to continued absorption of compound and redistribution into the plasma compartment. The free compound B is the same as in FIG. 10 and B1, B2, B3 and the numbers thereafter are as before; and

FIG. 12 is in two panels (12-1 and 12-2) that show the correlation of the free plasma concentration of compound B (ng/mL) with the percentage of inhibition of platelet aggregation in dogs treated by either infusion (12-1) or bolus (12-2).

### DETAILED DESCRIPTION OF THE INVENTION

The invention provides a process for restoring platelet aggregation following administration of a specified fibrinogen GPIIb/IIIa receptor antagonist compound. The process comprises administering to a patient in need thereof a therapeutic amount of antibody combining site-containing molecules (antibodies) that specifically bind to such fibrinogen GPIIb/IIIa receptor antagonists and inhibit the pharmacological activity of those antagonist molecules. Preferably, the antibody is administered to the patient in a therapeutically effective amount that provides a plasma level concentration that restores platelet aggregation to at least 50 percent within about 30 minutes following antibody administration. More preferably, the amount administered restores platelet aggregation to at least 50 percent in about 5 to about 15 minutes following antibody administration. These platelet aggregation times are measured ex vivo as discussed below.

A. Therapeutic Process

The present invention contemplates a process for restoring human or other mammalian platelet aggregation or adhesion to a host whose platelet aggregation time has been lengthened by administration of a specific class of GPIIb/IIIa receptor antagonist compound. In accordance with this process, a therapeutically effective amount of antibody combining site-containing molecules that specifically bind to (immunoreact with) a reversibly-bound GPIIb/IIIa receptor antagonist compound (GPIIb/IIIa antagonist) is administered to a host (human patient or other mammal) in need thereof. The contemplated antibody combining site-containing molecules specifically bind to a compound of the class of GPIIb/IIIa antagonist compounds that exhibit a plasma half-life of about two hours to about thirty-six hours and a GPIIb/IIIa receptor off-rate of about 0.07/seconds (t1/2˜1 seconds)to about 0.012/seconds (t1/2˜60 seconds). The antibody combining site-containing molecules described herein specifically bind to and inhibit the pharmacological activity of the GPIIb/IIIa antagonists.

The subject (host) is a mammal and, more preferably, a human patient being treated with a reversible GPIIb/IIIa antagonist compound for an ailment such as stroke, myocardial infarction, or unstable angina whether as an admitted patient to a hospital or as an ambulatory “out-patient”. The process can also be used in subjects undergoing operations to insert prostheses such as artificial heart valves or PCTA.

An effective therapy to mitigate the hemorrhagic events associated with the use of a GPIIb/IIIa receptor antiplatelet compound can be achieved by the use of antibody combining site-containing molecules that specifically bind to the antiplatelet compound and inhibit the pharmacological activity of the antiplatelet compound. These antibody combining site-containing molecules rapidly restore platelet aggregation and neutralize bleeding complications that can be associated with the administration of these GPIIb/IIIa receptor blockade agents. Thus, a contemplated process is particularly useful after the administration of a GPIIb/IIIa antagonist where hemorrhagic events are predicted to lead to excessive bleeding in 1 percent to 2 percent of the human patients that receive GPIIb/IIIa antagonist drugs, and where restoration of platelet aggregation and restored hemostatic function are desired.

Currently, GPIIb/IIIa antagonist compounds with off-rates less than about 0.009/seconds (t1/2˜75 seconds)and plasma half life greater than 2 hours are considered to be clinically “irreversible” because restoration of platelet aggregation in a clinically relevant time frame cannot be readily achieved by binding of the GPIIb/IIIa antagonist compound with another entity. This class of compounds also tends to have short plasma half-lives so that restoration of platelet aggregation after administration of such a compound is achieved by transfusion of further platelets from an exogenous source. In the event that a long plasma half-life is also associated with a compound with these kinetics no practical clinical reversal is possible.

The clinical irreversibility of the above types of GPIIb/IIIa antagonist compounds with the above off-rates and plasma half-lives is evidenced by the work reported by Reilly, T. M. et al., Ateriosclerosis, Thrombosis, and Vascular Biology, December, 1995, Vol. 15(12), p 2195-9. Reilly et al. reported studies with the cyclic peptide GPIIb/IIIa antagonist designated DMP 728 whose structure is shown hereinafter and monoclonal antibodies that bound to that molecule.

Those studies showed that DMP 728 infused into the femoral vein of anesthetized dogs at 20 mg/kg caused nearly complete inhibition of platelet aggregation for up to 210 minutes, measured ex vivo. When the monoclonal antibodies (at 0.2 or 1.0 mg/kg) were infused 10 minutes after the DMP 728, that inhibition of platelet aggregation was said to be attenuated by 50 percent at 3 hours. Although that amount of attenuation would be adequate if achieved in a short time, as is the case here, the three-hour time required to achieve that result is too long to be effective to treat and reverse a hemorrhagic event.

It is believed that the reason for that long time to achieve that reversal of inhibition of platelet aggregation lies in the slow off-rate and long plasma half-life exhibited by DMP 728 and similar compounds. On the other hand, the specific GPIIb/IIIa antagonist compounds and antibody combining site-containing molecules used here exhibit at least a 50 percent reduction in GPIIb/IIIa antagonist compound-induced inhibition of platelet aggregation in less than 30 minutes, and more usually in about 5 to about 15 minutes. Substantially complete reversal of that inhibition of platelet aggregation; i.e., about 90 to 100 percent reversal, is achieved here in about 60 minutes or less, measured ex vivo.

Platelet aggregation of human or other mammalian host such as a dog, sheep, horse, cattle, goat, mouse, rat, ape or monkey is typically determined by aggregation of platelet rich plasma after introduction of a platelet activating agent or agonist. The contribution of platelets to clot formation can also be measured by use of the activated clotting time (ACT) in the presence of heparin. A calculated ACT number is obtained by comparison of the clotting time of the blood of a subject treated with a GPIIb/IIIa antagonist drug to the standardized clotting time for a “normal” untreated animal with no detectable clotting defects, e.g., normal PT, aPTT, or platelet aggregation, of the same species; i.e., dog, mouse or human.

As used herein, the term “pharmaceutically effective amount” or “therapeutically effective amount” means an amount of antibody combining site-containing molecules that elicit the amount of restored platelet aggregation that is discussed before, as measured by ACT, and achieved within the times discussed before. The amount of restoration usually sought is at least 50 percent of the “normal” value.

Under ideal conditions, reversible binding between a ligand at one concentration, [L], and a receptor at the same or different concentration, [R], to form a ligand/receptor complex at some other concentration, [LR], typically follows a second order rate equation, having a forward reaction in which the ligand binds to the receptor and a reverse reaction in which the ligand and receptor separate. Both reactions have rate constants that are sometimes referred to as k₁ and k₋₁ or the on-rate and the off-rate, respectively. This reversible reaction is illustrated by the equation shown below

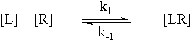

The concentration of ligand/receptor formed, [LR] is a function of the initial concentrations of ligand and receptor and the ratio k₁/k₋₁, or the equilibrium constant K_{eq}.

The drug ligand interaction with its biological receptor in a living organism is not an ideal condition, but the principles determined from ideal conditions can nevertheless be used for many drug ligand/receptor binding interactions. Here, a GPIIb/IIIa receptor antagonist compound can conveniently be grouped into one of three classifications by its binding ability to the GPIIb/IIIa receptor, or more easily, by rate of the reverse of the binding step, or the “off-rate”.

One group or class of GPIIb/IIIa antagonists binds so tightly that there is little reverse reaction, and those compounds exhibit an on-rate that is much greater than the off-rate. For many such compounds that are usually administered by i.v. infusion, restoration of platelet aggregation in a clinically relevant time frame cannot be readily achieved by binding of the GPIIb/IIIa antagonist compound with another entity. This class of compounds also tends to have short plasma half-lives so that restoration of platelet aggregation after administration of such a compound is achieved by transfusion of further platelets from an exogenous source. In the event that a long plasma half-life is also associated with a compound with these kinetics no practical clinical reversal is possible.

A second group or class of GPIIb/IIIa antagonists bind in such a way to the GPIIb/IIIa receptor that they exhibit an off-rate that is much faster than the first group or class of antagonists. When these compounds also have a short plasma half-life, they are normally administered intravenously, and restoration of platelet aggregation after administration of such a compound can be achieved by stopping the intravenous flow of the GPIIb/IIIa antagonist because of the relatively high off-rates exhibited by this class of compounds.

The third group of GPIIb/IIIa antagonists exhibit binding similar to the second class of antagonists. This class of antagonist demonstrates a longer plasma half-life. These GPIIb/IIIa antagonists are designed to be administered orally as a pill or capsule, or the like. The present invention is directed to this third class of GPIIb/IIIa antagonists that exhibit off-rates of about 0.7/seconds (t1/2˜1 second)to about 0.012/seconds (t1/2˜60 seconds).

Table 1 is provided below to illustrate compounds that are placed into the three classes of compounds discussed above. Table 1 illustrates the structures of several GPIIb/IIIa antagonist compounds and provides a classification of each based upon its off-rate in binding to the GPIIb/IIIa receptor. The “off-rates” for those compound classifications are: Class 1) 0.009/seconds (t1/2˜75 seconds) to 0.00012/seconds (t1/2˜6000 seconds), and short plasma half-life; Class 2) 0.7/seconds (t1/2˜1 second) to 0.012/seconds (t1/2˜60 seconds), and a short plasma half-life; and Class 3) 0.7/seconds (t1/2˜1 second) to 0.012/seconds (t1/2˜60 seconds), and a long plasma half-life. The current invention contemplates the use of the last class of compounds or those that have an off-rate of about 0.7/seconds (t1/2˜1 second) to 0.012/seconds (t1/2˜60 seconds), with the caveat that the plasma half-life of a contemplated antagonist compound is about two to about thirty-six hours.

TABLE 1
Classification of GPIIb/IIIa Antagonists by
Binding Off-Rate From the GPIIb/IIIa receptor
Classification: 1
Off-Rate: 0.009/seconds (t1/2˜75 seconds) to
0.00012/seconds (t1/2˜-6000 seconds) and a short plasma
half-life

DMP728

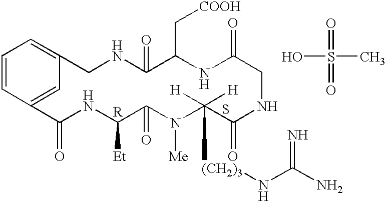

DMP 802

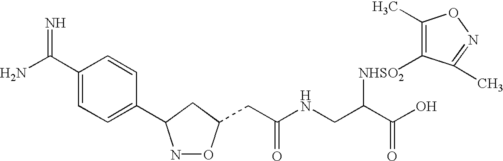

Aggrastat

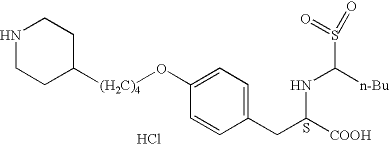

Roxifiban

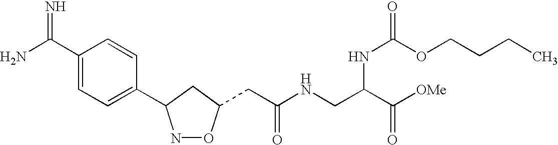

Classification: 2
Off-Rate: 0.7/seconds (t1/2˜1 second) to
0.012/seconds (t1/2˜60 seconds), and a short plasma
half-life

Fradafiban

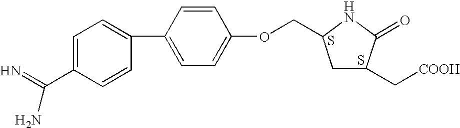

Lamifiban

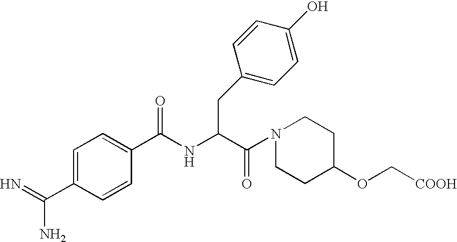

Classification: 3
Off-Rate: 0.7/seconds (t1/2˜1 second) to
0.012/seconds (t1/2˜60 seconds), and a long plasma half-
life

Compound B

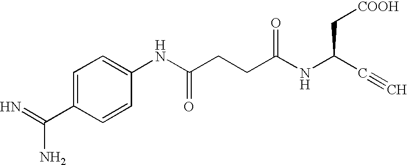

Compound D

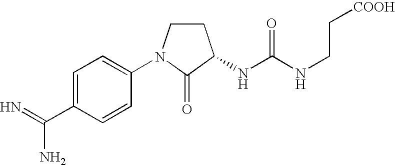

GR 144.053

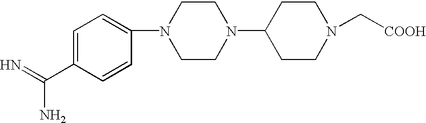

XR 299

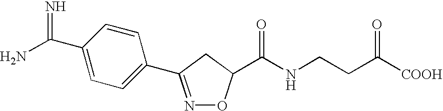

Sibafiban

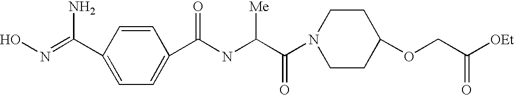

L 70314 *

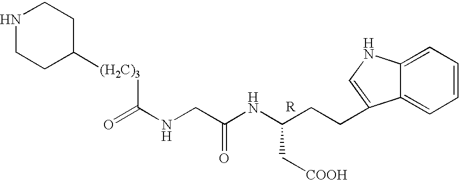

Lotrafiban *

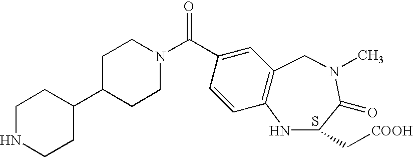

* R and S configurations are noted for L 70314 and Lotrafiban, respectively.

It is also contemplated that a GPIIb/IIIa antagonist compound be present as a pharmaceutically acceptable salt. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Suitable pharmaceutically-acceptable base addition salts of compounds of Formula I include metallic ion salts and organic ion salts. More preferred metallic ion salts include, but are not limited to appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by conventional means from the corresponding compound by reacting for example, the appropriate acid or base with the GPIIb/IIIa antagonist compound.

In a preferred embodiment of this process, the GPIIb/IIIa antagonist compound is a β-amino acid derivative described in U.S. Pat. No. 5,344,957, which disclosure is incorporated herein by reference. Those compounds have structures that correspond to the general formula:

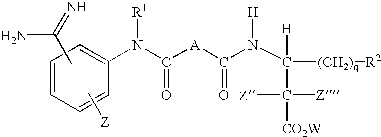

A particularly preferred embodiment of the compounds of the above disclosure is a compound referred to herein as “compound A” that corresponds in structure to the formula below and is named thereafter:

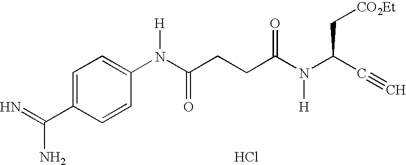

(ethyl 3S-[[4-[[4-(aminoiminomethyl)phenyl]-amino]-1,4-dioxobutyl]amino]-4-pentynoate monohydrochloride).

Compound A is the prodrug form of the active GPIIb/IIIa antagonist “compound B”, which corresponds in structure to the formula below and is named thereafter:

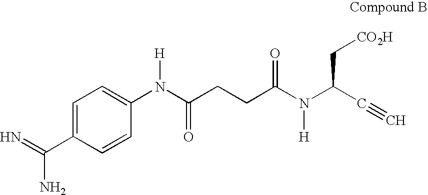

(3S-[[4-[[4-(aminoiminomethyl)phenyl]amino]-1,4-dioxobutyl]amino]-4-pentynoic acid).

In another preferred embodiment of a contemplated process, the antiplatelet GPIIb/IIIa antagonist compound is one of the 1-amidinophenyl-pyrrolidones, piperidinones or azetidinones described in PCT Application Publication No. WO 94/22820 (published Oct. 13, 1994), which disclosure is incorporated herein by reference. These compounds correspond in structure to the general formula:

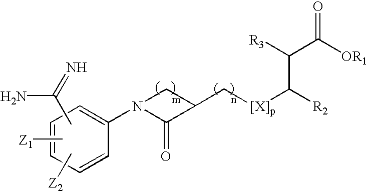

A particularly preferred embodiment of these compounds is “compound C” that corresponds in structure to the formula shown below and is named thereafter:

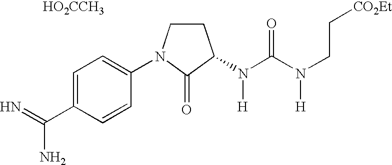

(N-[[[1-[4-(aminoiminomethyl)phenyl]-2-oxo-3S -pyrrolidinyl]amino]carbonyl] β-alanine, ethyl ester).

Compound C is the prodrug form of the active GPIIb/IIIa antagonist “compound D”, which corresponds in structure to the formula shown below that is named thereafter:

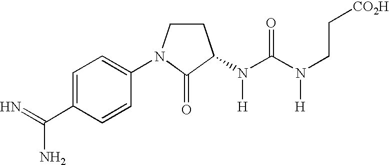

(3-[[[[1-[4-(aminoiminomethyl)phenyl]-2-oxo-pyrrolidin-3S-yl]amino]carbonyl]amino]propanoic acid).

At the present time, the most widely used compound to prevent blood clotting is the anticoagulant heparin. Heparin is a glycosaminoglycan that is found in human tissues that contain mast cells. The heparin administered to contemplated subjects is typically extracted from porcine intestinal mucosa or bovine lung.

Both compound B and D are removed from circulation almost exclusively by the kidney, and decreases in creatinine clearance due to renal impairment are associated with appreciable increases in the elimination half-life of the compounds. In renally impaired patients, reversal of the functionality of these compounds would be especially useful in an emergency situation where rapid removal of active compound is desired.

A process of this invention can be used on subjects being treated (i) only with a class 3 GPIIb/IIIa antagonist antiplatelet compound having the off-rate and plasma half-life properties discussed before (class 3), (ii) with a class 3 GPIIb/IIIa antagonist compound in combination with one or more additional antiplatelet compounds, or (iii) treated with a class 3 antiplatelet GPIIb/IIIa antagonist compound in combination with heparin. When the subject is being treated with a class 3 antiplatelet compound, above, without heparin, the invention also contemplates adding heparin to the sample prior to the measurement of the activated clotting times so as to enhance the effect of the antiplatelet compound.

In accordance with the invention, antibody combining site-containing molecules (antibodies) can be contacted with the blood of the host mammal to form antibody-treated blood ex vivo as in a dialysis machine, or more preferably in vivo in a living mammalian host. When administered to the host in vivo, the antibody combining site-containing molecules are preferably provided parenterally in one bolus injection, or taking up to 15 minutes, or more slowly as by i.v. infusion. The parenterally-provided antibodies can be administered intraperitoneally, intramuscularly, or intravenously.

Bolus or continuous (or continual) intravenous (i.v.) administration of an antibody-containing solution are the preferred route of administration of the antibodies to a host mammal. The i.v.-administered exogenous antibodies are present in the i.v. solution in an amount sufficient to provide a steady state plasma level concentration of those antibodies during the period of administration that achieves at least 50 percent restoration of ACT within 30 minutes of starting antibody administration.

Suitable intravenous compositions include bolus or extended infusion. Such intravenous compositions are well known to those of ordinary skill in the pharmaceutical arts. Those of skill in the art can readily determine the various parameters and conditions for administering the antibody without resort to undue experimentation.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The contemplated antibodies are administered in an amount sufficient to produce the desired therapeutic effect; i.e., the amelioration of the effect of the GPIIb/IIIa antagonist as discussed before. The dosage is not to be so large as to cause adverse side effects, such as hyperviscosity syndromes, pulmonary edema, conjestive heart failure, anaphylactoid reactions and the like. The dosage regimen utilizing the antibody is selected in accordance with a variety of factors including type, age, weight, sex, and medical condition of the patient; the route of administration; the renal and hepatic function of the patient; and the particular antibody employed. An ordinarily skilled physician or veterinarian can readily determine and prescribe the effective amount of antibody combining site-containing molecules required to restore platelet function based on the affinity of the particular antibody combining site-containing molecules for the GPIIb/IIIa antagonist compound used. The dosage can be adjusted by the individual physician in the event of any complication.

Typical dosages vary from about 0.1 mg/kg to about 25 mg/kg, preferably from about 1 mg/kg to about 10 mg/kg, most preferably from about 1 mg/kg to about 5 mg/kg, in one or more dose administrations daily. Intravenously, the most preferred dose is about 0.1 to about 5 mg/kg/minute during a constant or continual rate of infusion to provide a plasma level concentration during the period of time of administration of about 400 μg/mL and to about 500 μg/mL. The dosage objective is to achieve a therapeutic level of antibody that is sufficient to provide at least a 80 percent restoration of the ACT as measured against the standardized clotting time for a normal (clotting disease-free) untreated animal of the same species; i.e., dog, mouse or human.

Activated clotting times can be measured by several instruments presently available. The two most widely available instruments are the Hemotech™ (Medtronic, Parker Colo. U.S.A. 80134-9061) and the Hemochron™ (International Techindyne; N.J. U.S.A).

The Hemotech™ uses a mechanical plunger that is dipped in and out of kaolin-activated blood samples. Coagulation tests are performed using multiple two-channel test cartridges. Each cartridge contains a reagent reservoir and a reaction chamber and is either prewarmed in an external heat block or warmed in the instrument. The blood sample is added to the warmed cartridge.

When the test is initiated, the machine automatically empties the kaolin reagent into the reaction chamber and begins raising and lowering a plunger in each chamber at predetermined intervals. The action of the plunger mixes the sample with reagent and tests for clot formation. When a clot forms, the downward motion of the plunger is decreased. The decrease in the fall rate of the plunger is detected by a photo-optic system and the machine signals the formation of a clot. Individual clotting times, or the average and differences for the channels, are displayed on the front of the machine.

The Hemochron™ is a similar device that uses diatomaceous earth instead of kaolin to activate clotting of the blood. The Hemochron™ measures clot formation by monitoring a magnet as it moves away from the detector.

A comparison of the two instruments has shown that the measurements from each machine cannot be used interchangeably. A. Avendaco and J. Ferguson, J.A.C.C (Mar. 15, 1994) Vol. 23 (4) pp. 907-910. Thus, a standardized concentration curve is also standardized for available testing equipment.

The invention provides a process for restoring platelet aggregation following administration of a specified class 3 fibrinogen GPIIb/IIIa receptor antagonist compound. The process comprises administering to a patient in need thereof a therapeutic amount of antibody combining site-containing molecules (antibodies) that specifically bind to such fibrinogen GPIIb/IIIa receptor antagonists and inhibit the pharmacological activity of those antagonist molecules. Preferably, the antibody is administered to the patient in a therapeutically effective amount that provides a plasma level concentration that restores platelet aggregation to at least 50 percent within about 30 minutes following antibody administration. More preferably, the amount administered restores platelet aggregation to at least 50 percent in about 5 to about 15 minutes following antibody administration. These platelet aggregation times are measured ex vivo as discussed before.

In one preferred embodiment, the GPIIb/IIIa receptor antagonist compound is 3S-[[4-[[4-(aminoiminomethyl)phenyl]-amino]-1,4-dioxobutyl]amino]-4-pentynoic acid (compound B); or (3-[[[[1-[4-(aminoiminomethyl)phenyl]-2-oxo-pyrrolidin-3S-yl]amino]carbonyl]amino]propanoic acid (compound D); and the reagent that immunoreacts with the antiplatelet compound is a monoclonal antibody.

Additionally preferred are monoclonal antibodies that bind to derivatives of compounds A, B C, or D, for example esters or salts, as disclosed in U.S. Pat. Nos. 5,344,957 and 5,721,366, or metabolites of compounds A, B, C, or D. Preferred monoclonal antibodies are an antibody secreted by hybridomas designated ATCC HB-12081 and HB-12082.

More preferably, the antibody combining site-containing molecules that immunoreact with one or the other or both of compounds B and D are polyclonal antibodies. Such polyclonal antibodies or antibody combining site-containing portions are obtained from large mammals such as sheep, horses or cattle. Useful antibody combining site-containing molecules are obtained as discussed hereinafter.

B. Antibodies

In the practice of any of the above-described processes, the reagent that immunoreacts with the antiplatelet compound can be either polyclonal antibodies (antiserum) or monoclonal antibodies. In one preferred embodiment, the reagent that immunoreacts with the antiplatelet compound is a monoclonal antibody, whereas in another preferred embodiment that reagent comprises polyclonal antibodies.

The term “antibody” in its various grammatical forms is used herein to refer to immunoglobulin molecules and immunologically active portions of immunoglobulin molecules; i.e., molecules that contain an antibody combining site or “paratope”. “Antibody” as used herein can refer to intact immunoglobulin molecules or any portions of an immunoglobulin molecule that contain the paratope, including those portions known in the art as Fab, Fab′, F(ab′)₂, F(v), and single chain antibodies generated by phage display [SC F(v)]. When whole antibodies are used, it is preferred that the antibodies be of the IgG class as compared to being of the IgM, IgA, IgD or IgE class.

The term “immunoreact” in its various forms refers to the specific binding between an antigenic determinant-containing molecule and a molecule containing an antibody combining site such as a whole antibody molecule or a portion thereof. The term “antigenic determinant” refers to the actual structural portion of the antigen that is immunologically bound by an antibody combining site. The term is also used interchangeably with “epitope”. As used herein, the term “specific binding” in its various forms refers to a non-random binding reaction between a cell surface receptor and a ligand molecule.

The word “immunogen” is used herein to mean the chemical entity that induces production of antibodies, whereas the word “antigen” is used for the chemical entity that is bound by the antibodies. An immunogen is almost always an antigen, but an antigen need not be an immunogen.

Some molecules do not induce an immune response when used as an immunogen. However, linkage of those same molecules to a carrier molecule to form a conjugate or imunoconjugate, and immunization of a mammal with the conjugate can induce production of antibodies that immunoreact with the immunogen. Such molecules that are not immunogenic when used alone and are immunogenic when bonded to a carrier molecule to form a conjugate are referred to in the art and herein as hapten molecules.

The contemplated platelet GPIIb/IIIa receptor antagonist molecules that exhibit an off-rate of about 1 second to about 60 seconds and a plasma half-life of about two to about thirty-six hours (class 3 compounds) typically do not themselves induce the production of antibodies when used to immunize a mammal. Those compounds can, however, be linked to a carrier molecule to form a conjugate as discussed hereinbefore, and be used successfully as such a conjugate to induce production of antibodies in an immunized mammal. The compounds are thus haptens.

Examination of the structural formulas in Table 1 for the contemplated class 3 antagonist compounds illustrates that each has a carboxyl and/or an amino group that can be utilized to link the antagonist compound to the carrier molecule. The examples that follow illustrate such linkages and use of the resulting conjugates to induce production of useful antibody combining site-containing molecules.

Polyclonal antibodies or “antisera” can be produced by injecting a mammal, for example a goat, mouse, sheep or rabbit, with the compound to which the antibodies are to be raised; i.e., the immunogen. When the antibody level or “titer” reaches a sufficient level, antibody-containing serum is drawn from the animal. Antibodies that immunoreact with an antigen of interest such as the immunogen can be separated by techniques known to those skilled in the art such as by affinity chromatography.

Monoclonal antibodies can be produced using known processes such as that described by Kohler and Milstein in Nature, Vol. 256: 495-497 (1975), the text of which is incorporated herein by reference. Generally, a mouse is inoculated with an immunogen of interest. This innoculation stimulates the proliferation of lymphocytes expressing antibodies against the immunogen. Lymphocytes are taken from the spleen and fused to myeloma cells by treatment with a polymer such as polyethylene glycol. Hybrid cells are selected by growing in a culture medium that does not permit the growth of unfused cells. Individual hybrid cells are further cultured and tested for the presence of antibodies that bind the immunogen, when used as an antigen.

In an additional embodiment, monoclonal antibodies produced in germ-free animals are utilized, following the disclosures of PCT/US90/02545. According to another embodiment of the invention, human antibodies can be used and can be obtained by using human hybridomas (Cote et al., 1983, Proc. Natl. Acad. Sci. U.S.A. 80:2026-2030) or by transforming human B cells with Epstein-Barr virus in vitro (Cole et al., 1985, in Monoclonal Antibodies and Cancer Therapy, Alan R. Liss, pp. 77-96.

In fact, techniques developed for the production of “chimeric antibodies” or “humanized antibodies” (Morrison et al., 1984, J. Bacteriol. 159-870; Neuberger et al., 1984, Nature 312:604-608; Takeda et al., 1985, Nature 314:452-454) by splicing the genes for a mouse antibody molecule useful in the present invention together with genes from a human antibody molecule of appropriate biological activity can be used.

Contemplated chimeric antibodies are those that contain a human Fc portion and a murine (or other non-human) Fv portion. Contemplated humanized antibodies are those in which the murine (or other non-human) complementarity determining regions (CDR) are incorporated in a human antibody; i.e., an antibody whose protein sequence is that of a human antibody. Both chimeric and humanized antibodies are monoclonal. Such chimeric human or humanized antibodies are preferred for use in in vivo therapy, because the chimeric human or humanized antibodies are much less likely than xenogeneic antibodies to induce an immune response, in particular an allergic response.

Another embodiment is the production of single chain antibodies from a phage display library. In this embodiment, antibody variable domains or V genes are cloned from populations of lymphocytes and expressed in a filamentous bacteriophage. The phage display the heavy and light chain variable domains on their surface and selection of more specific and potent antigen recognition can be achieved by successively mutating the phage (Winter et al., 1994, in Annual Reviews of Immunology, 12:433-455).

In certain instances, the immunogen of interest does not stimulate the inoculated mammal to produce antibodies. In order to ensure production of monoclonal or polyclonal antibodies, the immunogen is bonded to a carrier molecule to produce a conjugate compound large enough to stimulate an immune response in the animal.

Carrier molecules typically comprise a protein, for example bovine serum albumin (BSA), thyroglobulin, HBcAg, tetanus toxoid or keyhole limpet hemocyanin (KLH). An immunogenic polypeptide with a length of about 15 to about 70 amino acid residues and having the sequence from about position 70 through about position 140 from the amino-terminus of HBcAg can also be used as the carrier molecule as is disclosed in U.S. Pat. No. 4,818,527. A synthetic carrier such as the branched ologolysine described in Tam et al., 1989, Proc. Natl. Acad. Sci. USA, 86:9084-9088 or the similarly prepared brancehd olioglysine that is also linked to resin particles as described in Butz et al., 1994, Pep. Res., 7(1):20-23 can also be used.

The new compounds, comprising the carrier molecule bonded to an immunogen of interest, are known as conjugates or immunoconjugates and can be prepared by processes known to those skilled in the art.

As noted elsewhere, the use of antibody combining site-containing molecules is contemplated here, and it is noted here that as is well known, an antibody combining site can be well mimicked by so-called single chain antibodies. As a consequence, procedures described for the production of single chain antibodies (U.S. Pat. No. 4,946,778) can be adapted to provide single chain antibodies that are useful in the present invention.

Antibody fragments that contain the idiotype (paratope or combining site) of the antibody molecule can be prepared by known techniques. For example, such fragments include but are not limited to: the F(ab′)₂ fragment that can be produced by pepsin digestion of the antibody molecule; the Fab′ fragments that can be prepared by reducing the disulfide bridges of the F(ab′)₂ fragment; and the Fab fragments that can be prepared by treating the antibody molecule with papain.

Such antibody fragments can be prepared from any of the polyclonal or monoclonal antibodies of the invention. Exemplary antibody fragments are prepared using monoclonal antibodies produced by a hybridoma designated ATCC HB-12081 or HB-12082.

An additional embodiment of the invention utilizes the techniques described for the construction of Fab expression libraries (Huse et al., 1989, Science 246:1275-1281) to permit rapid and easy identification of monoclonal Fab fragments with the desired specificity for an antibody useful in the present invention.

In the production of antibodies, screening for the desired antibody can be accomplished by techniques known in the art, e.g., radioimmunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, and the like. In one embodiment, antibody binding is detected by use of a label on the primary antibody. In another embodiment, the primary antibody is itself detected by the binding of a secondary antibody or other reagent such as protein A to the primary antibody. In a further embodiment, the secondary antibody is labeled. Many means are known in the art for detecting binding in an immunoassay and are within the scope of the present invention.

The following hybridomas, which produce monoclonal antibodies preferred for the practice of this invention, were deposited with the American Type Culture Collection (ATCC) 10801 University Boulevard, Manassas, Va., 20110-2209, USA on Apr. 12, 1996:

1) P187.4D7.B3.A1 (also referred to herein as “4D7”) assigned ATTC Accession No. HB-12081; and

2) P187.9F7.A5.A1 (also referred to herein as “9F7”) assigned ATTC Accession No. HB-12082.

The hybridomas were deposited under conditions that assure that access to the hybridoma will be restricted during the pendency of the patent application, and that all restrictions on the availability to the public of the hybridoma as deposited will be irrevocably removed upon the granting of the patent.

Thus, this invention provides an antibody that immunoreacts with any of the particular antiplatelet compounds (class 3) described above. Inasmuch as the antibodies are induced by a haptenic form of a class 3 antagonist molecule, those antibodies do not immunoreact with heparin that can be present in a subject's blood. The antibodies can be purified or unpurified polyclonal antisera or monoclonal antibodies, including immunoreactive fragments thereof. This invention also provides hybridomas that produce or “secrete” the monoclonal antibodies. In a preferred embodiment, this invention also provides a monoclonal antibody, produced by a murine hybridoma cell line that immunoreacts with an antiplatelet compound and essentially does not immunoreact with heparin.

C. Kits

It is anticipated that the antibodies of the invention will be provided as part of a “kit” for performing the processes of the invention. The kit provides an immunoreactive “reagent”; i.e., the antibodies defined above, for a specific antiplatelet compound. Typically, the kit also includes a set of instructions for use.

Thus, the invention also provides a kit comprising a reagent that immunoreacts with and reverses the activity of a particular platelet GPIIb/IIIa receptor antagonist compound, and thus provides a means for restoring the rate of platelet aggregation in a subject being treated with the compound. In a preferred embodiment of the kit, the reagent comprises a monoclonal antibody produced by the hybridoma designated ATCC HB-12081. In a separately preferred embodiment of the kit, the reagent comprises a monoclonal antibody produced by the hybridoma designated at the ATCC HB-12082. In yet another preferred kit, the reagent comprises polyclonal antiserum that immunoreacts with compound B. In still another embodiment, the reagent comprises polyclonal antiserum that immunoreacts with compound D.

The above-described invention is further illustrated in the following Examples. These Examples are not intended, nor should they be interpreted, to limit the scope of the invention, which is more fully defined in the claims that follow.

### EXAMPLE 1

### Measuring Activated Clotting Times (ACT) in Heparinized Human Whole Blood

This example shows how activated clotting times were measured using a Hemochron™-8000. This process can be used to calculate a standardized concentration curve for various known concentrations of anti-platelet compounds. The process can also be used to measure activated clotting times for use in calculating the ACT number for the processes of the invention.

1. Preparation

A. Blood was drawn from a human volunteer into a syringe containing heparin. The heparin to whole blood ratio was 1:10 such that the final concentration of heparin in the syringe was 1.4 Units/mL.

B. The Hemochron™-8000 was set up to run ACT using the P-215 tubes from the same manufacturer (International Technidyne).

2. Procedure

A. A 1 mL sample of the above heparinized blood was admixed and maintained (incubated) with 2.5-10 μl of antagonist compound or saline for 5 minutes at room temperature.

B. The Hemochron™ was started as 400 μl of the test sample was pipetted into the P-215 tube. The tube contents were gently mixed, placed into the Hemochron™ and turned one revolution until the light on the instrument came on.

C. Step B was repeated for the second channel of the instrument within 30 seconds to give an average activated clotting time. The instrument detects clot formation and displays the time for each channel, as well as the average for the two.

D. When an antibody was used, it was added after the incubation of the compound with the heparinized whole blood and permitted to incubate for an additional 5 minutes before the test was started. Steps 2 and 3 were repeated as stated above.

The clotting time for each concentration or GPIIb/IIIa antagonist is divided by the clotting time for the control sample, i.e., no antagonist added. This ratio is the basis for constructing a standard curve for the antagonist. As shown by our data, the clotting time for therapeutically relevant concentrations of the antagonist will be the same as the control when the antibody is present in the assay.

In a patient, the assay would be run in two separate tubes or cartridges. One tube or cartridge would contain the antibody and the other would not. In the case of the non-heparinized patient both tubes or cartridges would also contain 1.4 Units/ml heparin. The clotting time for patients blood would be determined with both tubes or cartridges and the clotting time for the test without antibody would be divided by the clotting time in the presence of antibody to give the ratio, as above. The concentration of GPIIb/IIIa antagonist would then be determined from the concentration curve determined above.

In a patient to be administered antibody to reverse the therapeutic effect of the GPIIb/IIIa antagonist, a clotting time would be determined for the patient's blood before administration of the antibody. This clotting time would be determined with 1.4 Units/ml heparin in the assay. After administration of the antibody as a reversal agent, the clotting time would again be measured in the presence of 1.4 Units/ml heparin to ascertain whether the antibody had reversed the pharmacodynamic effect of the GPIIb/IIIa antagonist, i.e., the increase in clotting time.

### EXAMPLE 2

### Effect of Antiplatelet Compound Concentration on Activated Clotting Times

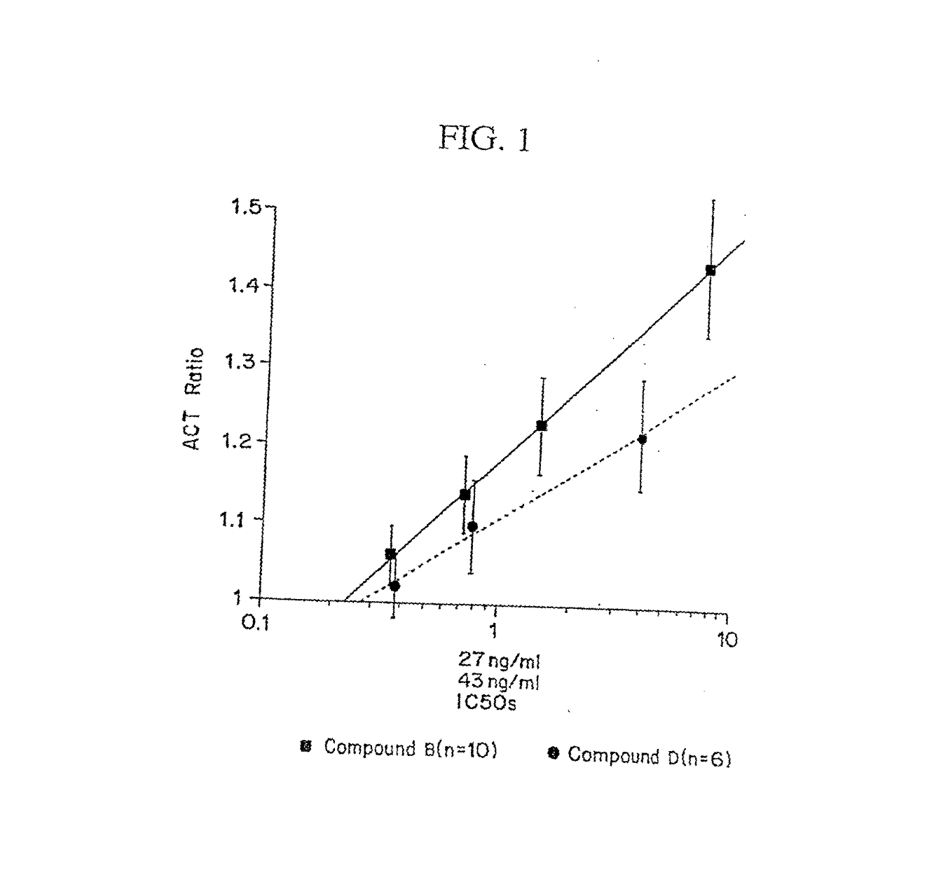

The activated clotting time was first measured (in seconds) for control whole blood from each donor containing only 1.4 Units/mL of heparin. The clotting times were then measured as in Example 1 by varying concentrations of antiplatelet compounds B and D in addition to heparin for each donor. The clotting time for a particular concentration of antagonist compound was divided by the clotting time of the control without any antagonist compound to provide the clot ratio. This clot ratio was then plotted against the compound concentration. FIG. 1 shows that different concentrations of antiplatelet compounds directly affect the activated clotting time as measured by the process of Example 1.

### EXAMPLE 3

### Effect of Polyclonal Antibodies on Activated Clotting Times

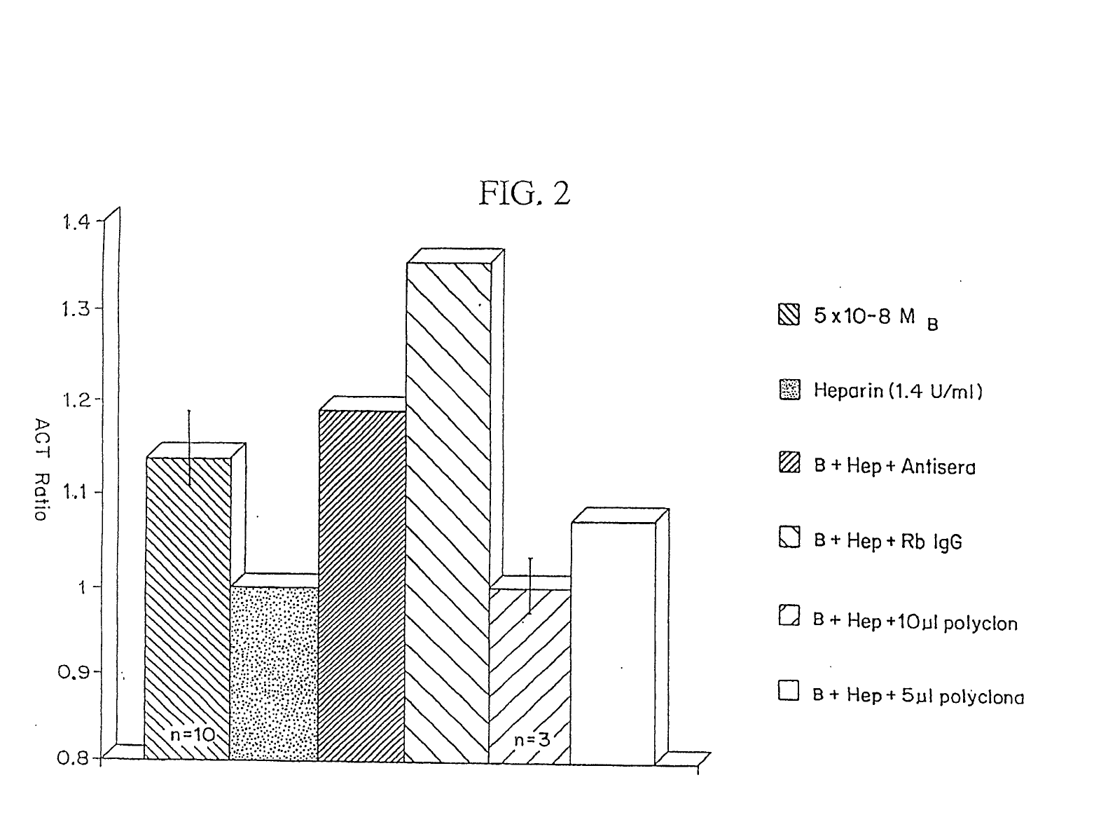

Activated clotting times were calculated as described in Example 1 for blood samples containing heparin only (control) and heparin with 5×10⁻⁸ M antiplatelet compound B in the absence and presence of irrelevant rabbit antisera, rabbit IgG, and 10 μl and 5 μl of a rabbit polyclonal antibody raised to compound B. The activated clotting time ratios were calculated as described in Example 2. As can be seen in FIG. 2, the presence of antibodies had a reversing effect on the activated clotting time of blood containing the antiplatelet compound. The higher concentration of antibody (10 μl) completely reversed the effect of compound B on the clotting time.

### EXAMPLE 4

### Monoclonal Antibody (Mab) Production

A lysine containing derivative of compound B, “compound E” (N-[N-[4-[[4-aminoiminomethyl)phenyl]-amino]-1,4- dioxobutyl]-L-aspartyl]-L-lysine,bis (trifluoroacetate), dihydrate), was used as the hapten for production of antibodies. This hapten was conjugated to thyroglobulin as carrier protein to provide a conjugate, and the resulting conjugate was used to immunize mice. The mice were screened for antibodies to compound E conjugated to bovine serum albumin (BSA) as antigen, and a mouse with the highest titer was chosen to produce clones for monoclonal antibody production.

Fusions and monoclonal antibody production were performed using standard techniques. Thus, Balb/c mice were immunized monthly via intraperitoneal injection of 25 μg of compound E. The immunogen was administered in Freund's adjuvant and the course of immunizations lasted eight weeks.

The spleen was excised from a mouse producing high titers of circulating anti-compound E antibodies and the spleen was dissociated to liberate splenocytes. The splenocytes were fused to mouse myeloma cells (SP2/mil6) obtained from American type Culture Collection, Rockville, Md. 20852, USA; ATCC No. CRL- 2016. See, J. Immunol. Methods, Vol. 148, pp. 199-207 (1992). The cells were fused with polyethylene glycol and grown under selective conditions (HAT medium) that permit only cells resulting from the fusion of a splenocyte with a myeloma cell to proliferate.

Progeny from the fusion were analyzed for the presence of antibodies by assessing the ability of conditioned media samples to bind immobilized compound E conjugated to bovine serum albumin. Positive progeny were subcloned into soft agar in order to obtain colonies of cells arising from the product of a single fusion event. Ten positive colonies were obtained that produced anti-compound E antibodies. Of the ten, nine were IgG1, K isotype and one (7C4) was IgG2, K isotype. All purified antibodies specifically bound compound B.

Ascites fluid containing these antibodies was produced in Balb/c mice and the antibodies were purified to homogeneity via Protein G-Sepharose affinity chromatography. Both the ascites and purified IgG were subsequently assayed in the aggregation assay (Example 5) and ACT assay (Example 7) for neutralizing activity.

### EXAMPLE 5

### Aggregation of Human Platelet Rich Plasma

Human platelet rich plasma (“PRP”) was prepared by centrifugation of citrated whole blood at 970×g for 3.5 minutes at room temperature. PRP was carefully removed from red cells and placed in 50 mL conical tubes. Platelet aggregation was measured as an increase in light transmission in an aggregometer (Bio/Data model PAP-4, Horsham, Pa.) using ADP (20 μM) or collagen (4 μg/mL) as the agonist.

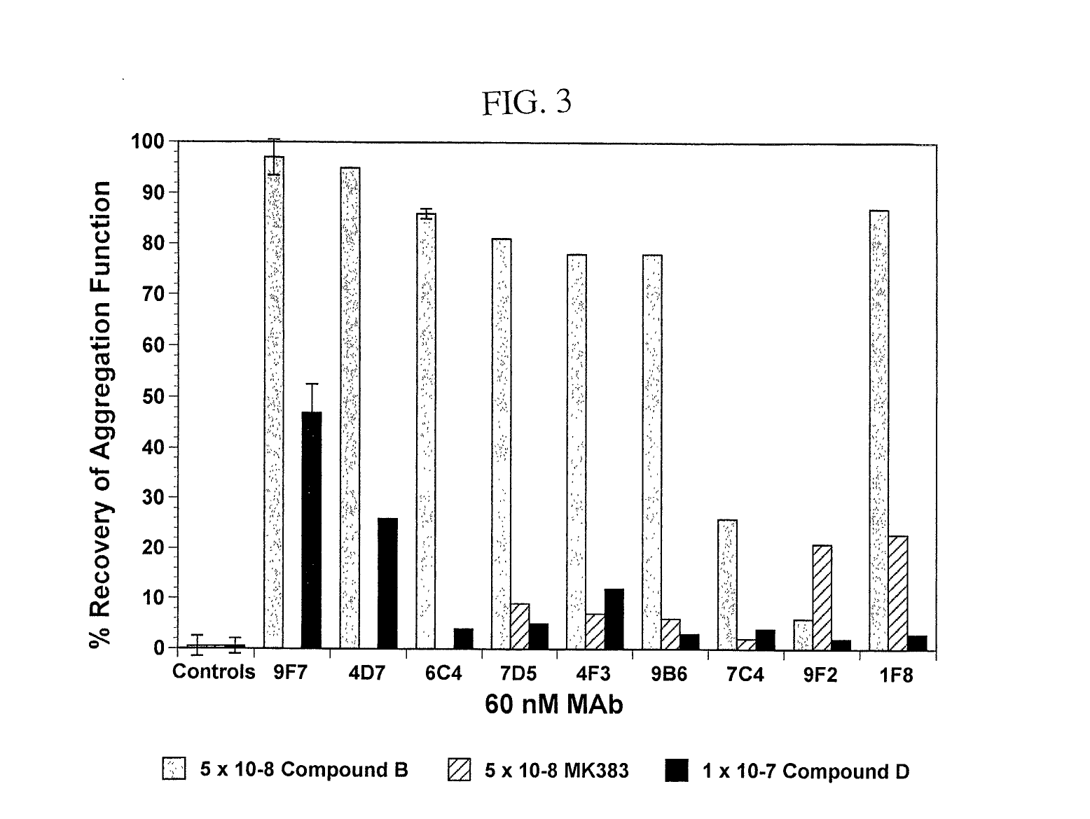

Antibodies produced according to Example 4 were assayed in aggregation for their ability to neutralize (at 60 nM) the GPIIb/IIIa antagonists compound B (5×10⁻⁸ M), compound D (1×10⁻⁷ M), and Merck compound MK383 (5×10⁻⁸ M), as a means of screening which ones recognized the antagonists in a useful manner. The levels of antiplatelet compounds used provided at least 50 percent inhibition of aggregation by themselves. Results are illustrated in FIG. 3.

### EXAMPLE 6

### Effect of Monoclonal Antibodies on the ACT of Compound B-treated Blood

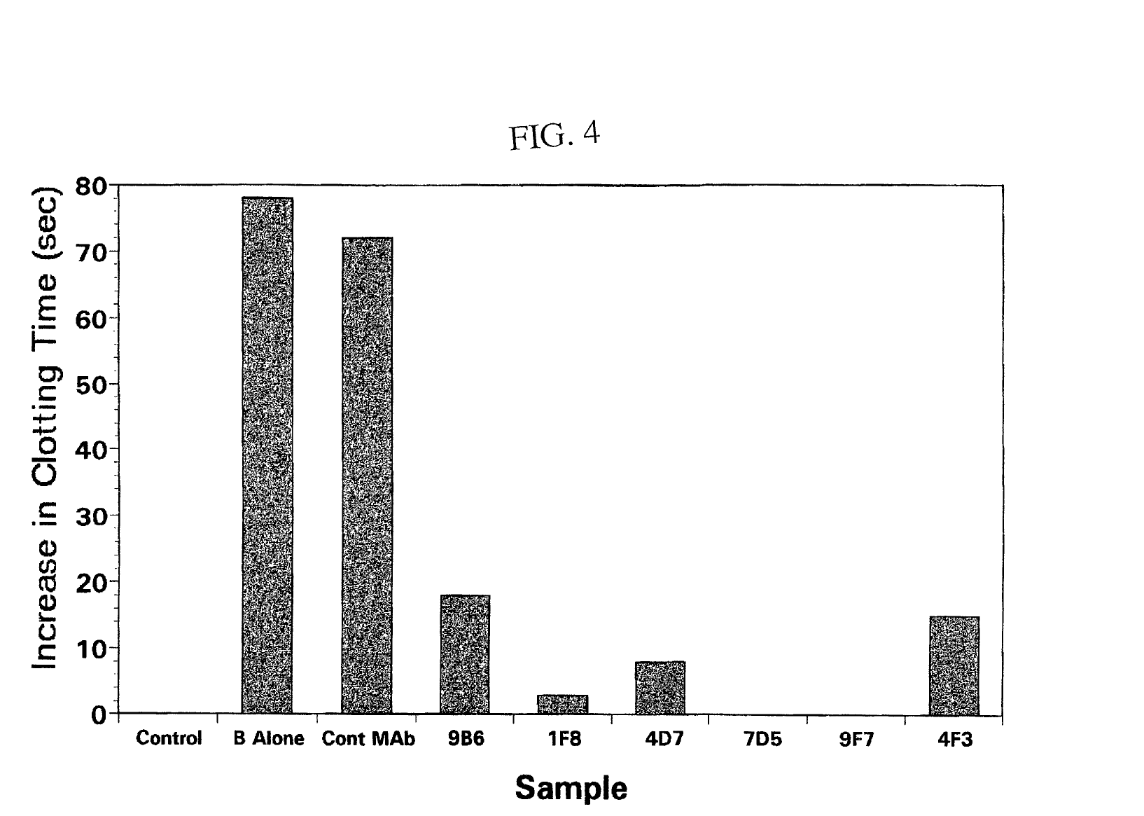

Several monoclonal antibodies were prepared as described in Example 4. Activated clotting times (ACTs) were calculated according to the process of Example 1 for a series of the antibodies in the presence of 5×10⁻⁸ M compound B. As seen from FIG. 4, all of the monoclonal antibodies lowered the clotting time as compared to that with compound B alone. A control monoclonal antibody (“Cont Mab”) against an irrelevant protein had no effect.

### EXAMPLE 7

### Neutralizing Activity of Monoclonal Antibody Molecules

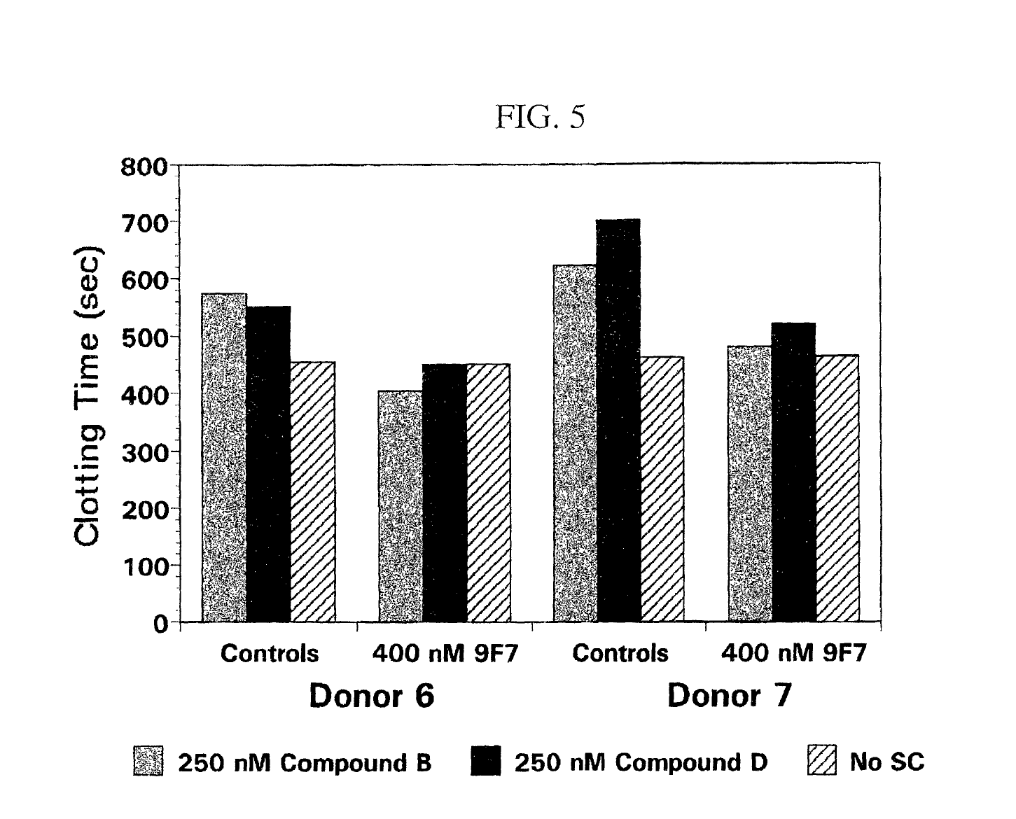

A monoclonal antibody produced according to Example 4, and coded monoclonal antibody (Mab) 9F7, was assayed at 400 nM in whole blood from two different donors for its ability to neutralize antiplatelet compounds B and D at 250 nM each. Activated clotting time measurements were made using a Medtronic Hemotec™ instrument. As seen in FIG. 5, antibody Mab 9F7 showed a distinct effect on the measured activated clotting times for each of the two antiplatelet compounds, with a greater effect being shown with compound B.

### EXAMPLE 8

### In vitro Platelet Aggregation Using Goat or Sheep Polyclonal Antibodies

Polyclonal antisera were raised in Alpine and Nubian goats immunized with a combination of immunoconjugates prepared from the thyroglobulin-conjugated compound B analogue discussed before and a conjugate prepared from KLH as carrier and Compound D. Semi-purified antibody preparations (non-affinity purified) from each animal were able to dose-relatedly reverse the activity of compounds B or D, appropriately, in a platelet-rich plasma assay. Two sheep were similarly immunized and their crude antibody preparation provided similar results.

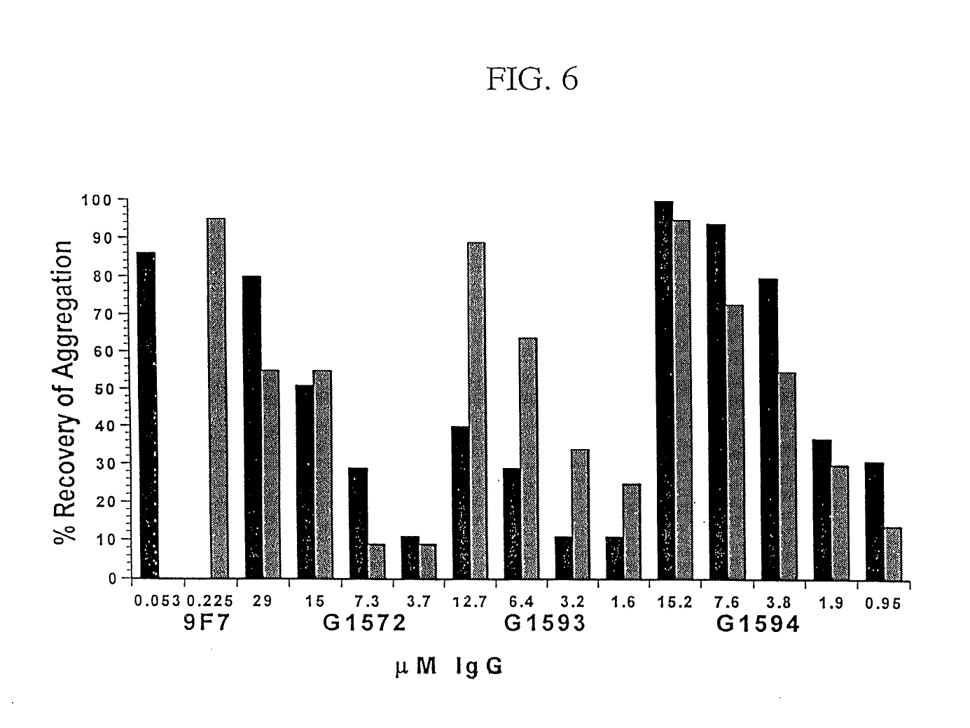

Data for polyclonal antibodies from three goats (G1572, G1593 and G1594) are shown in FIG. 6 as compared to data obtained using Mab 9F7, and in which compound B was added at 50 M (black bars) or compound D added at 100 nM (gray bars). Numbers between the bottom of the graph and the antibody designations are the micromolar (μM) concentrations of antibody molecules.

### EXAMPLE 9

### In vitro Platelet Aggregation

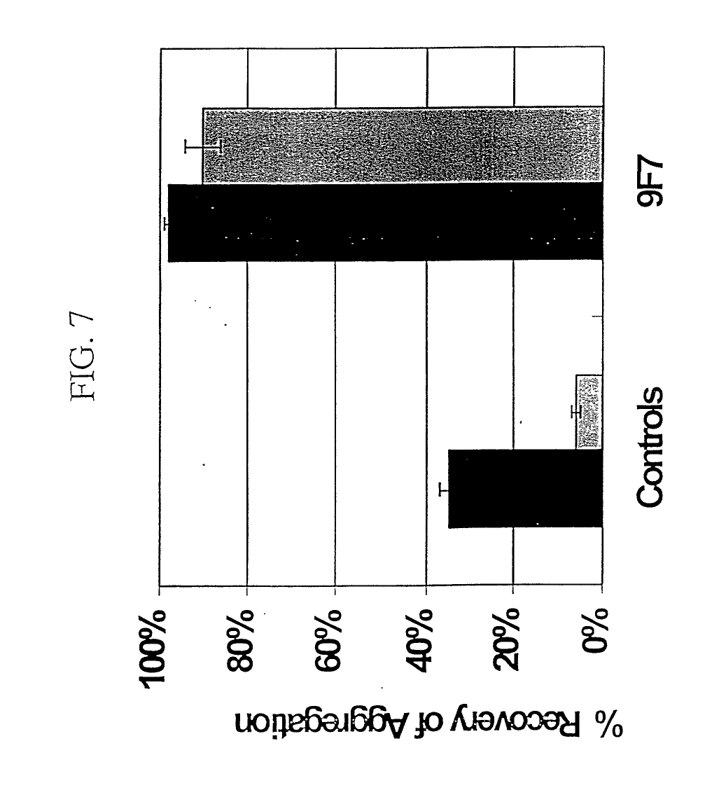

Collagen-induced platelet aggregation was measured in the presence of 50 nM compound B or 100 nM compound D over 3 minutes in the presence or absence of Mab 9F7 inclusion at 60 nM. As shown in FIG. 7, Mab 9F7 is a potent neutralizing monoclonal antibody that restores platelet activity in the presence of nearly fully inhibitory doses of compound B. In vitro, 60 nM antibody neutralized the effects of 50 nM of compound B (black bar) and nearly completely neutralized the effects of 100 nM B (gray bar) in the aggregation assay. Therefore 9F7 appears to inhibit B in an equimolar manner.

### EXAMPLE 10

### In vivo Platelet Aggregation

Guinea pigs were dosed either i.v. (compound B) or orally (compound A) until steady state platelet inhibition was reached. In the i.v. study, drug infusion was stopped prior to a 60 minute Mab 9F7 infusion and in the oral study a 15 minute Mab 9F7 infusion (1.67 μg/ml infusion for 60 minutes) was started about 30 minutes after the last oral dose. Blood samples were collected at 20, 40 and 60 minutes (i.v. study) and at 5, 10 and 15 minutes (oral study) during 9F7 infusion to measure platelet aggregation.

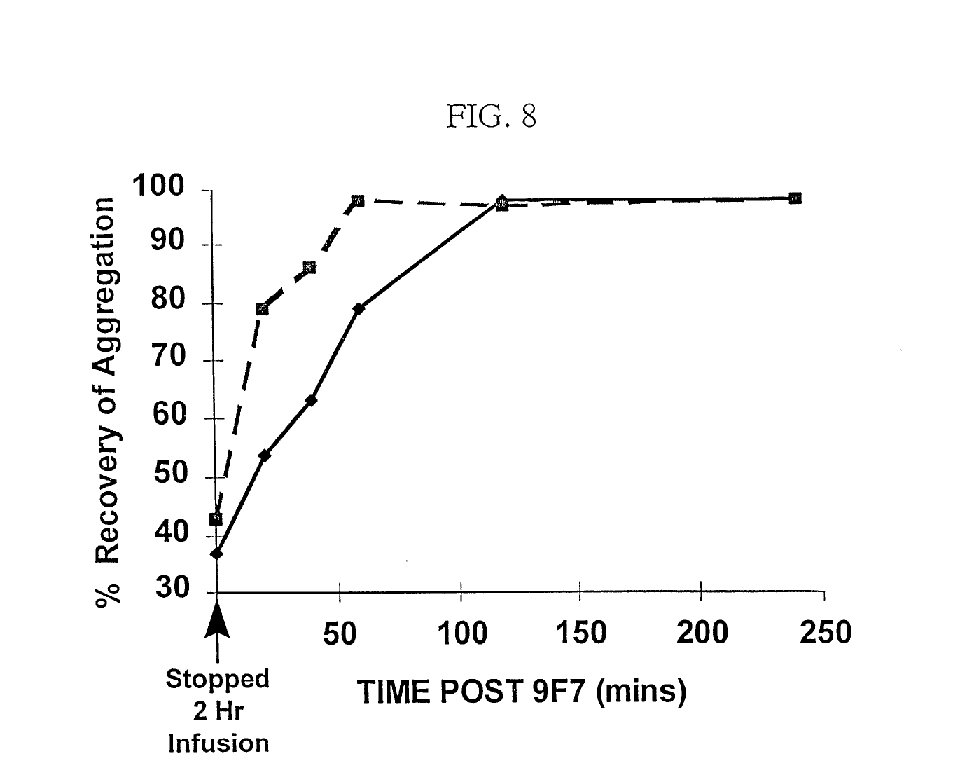

It was found that Mab 9F7 treatment rapidly restored platelet aggregation (FIG. 8). However, the guinea pig has limited utility as an efficacy model due to the small volume of blood that can be sampled and the short half-life of compound B in this species. Therefore, follow-up studies were conducted in dogs because the half-life of compounds B in this species is more similar to the human half-life than is the half-life in the guinea pig.

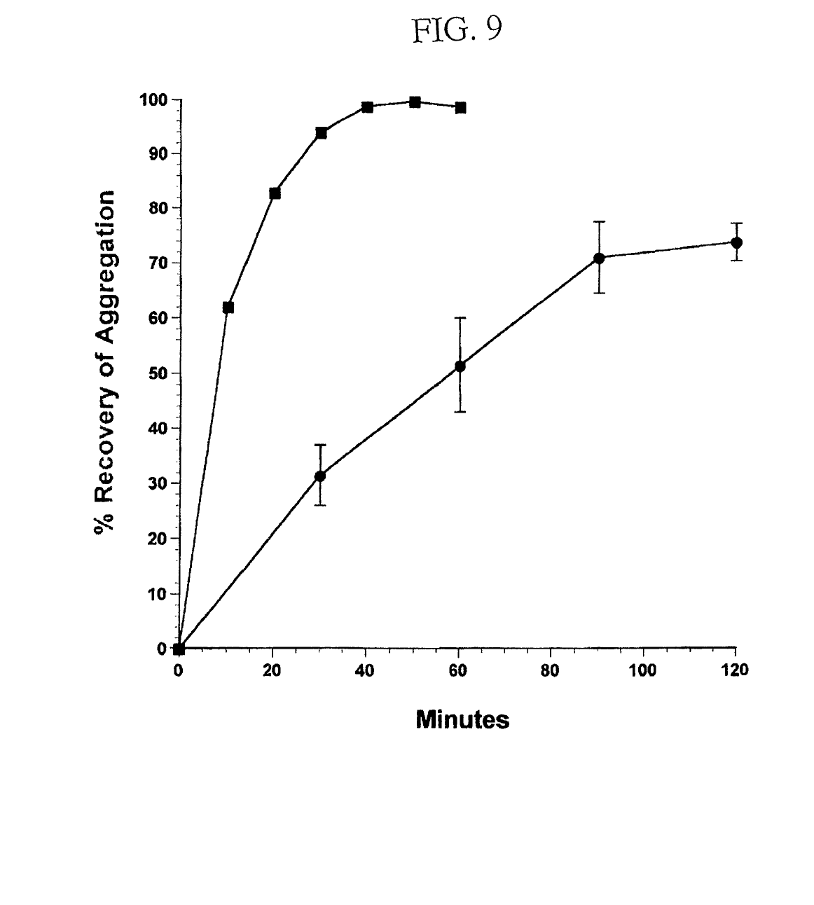

Three beagle dogs were therefore infused with compound B while monitoring blood pressure, heart rate, PT (prothrombin time), and aPTT (activated partial thromboplastin time). At the 2 hour time point, the drug infusion was terminated and blood samples were drawn at 10 minute intervals for the next hour as Mab 9F7 was infused at the rate of 1.67 mg/min (0.5 mL/min) in one of the dogs. The results from this study showed that the ex vivo aggregation response was restored more quickly with Mab 9F7 present (FIG. 9) than when that monoclonal antibody was absent.

In a second study, a dog was treated with a capsule of 10 mg of compound A, BID for 4 days (15 mg on day 3) prior to antibody testing. This level of dosing resulted in a 54 percent inhibition of platelet function. Four hours after the last oral dose of compound A, a dose of pentobarbitol and the monoclional antibody (P187-9F7) was infused by a i.v. bolus injection. Each bolus contained a 5-fold molar excess over drug plasma levels (about 50 mg of Mab 9F7). A control dog was given identical amounts of antibody without compound A oral dosing. Blood samples were collected for aggregation assays at 5, 15, 30 and 60 minutes after each bolus administration of antibody, plasma levels of Mab 9F7, and total and free plasma levels of compound B.

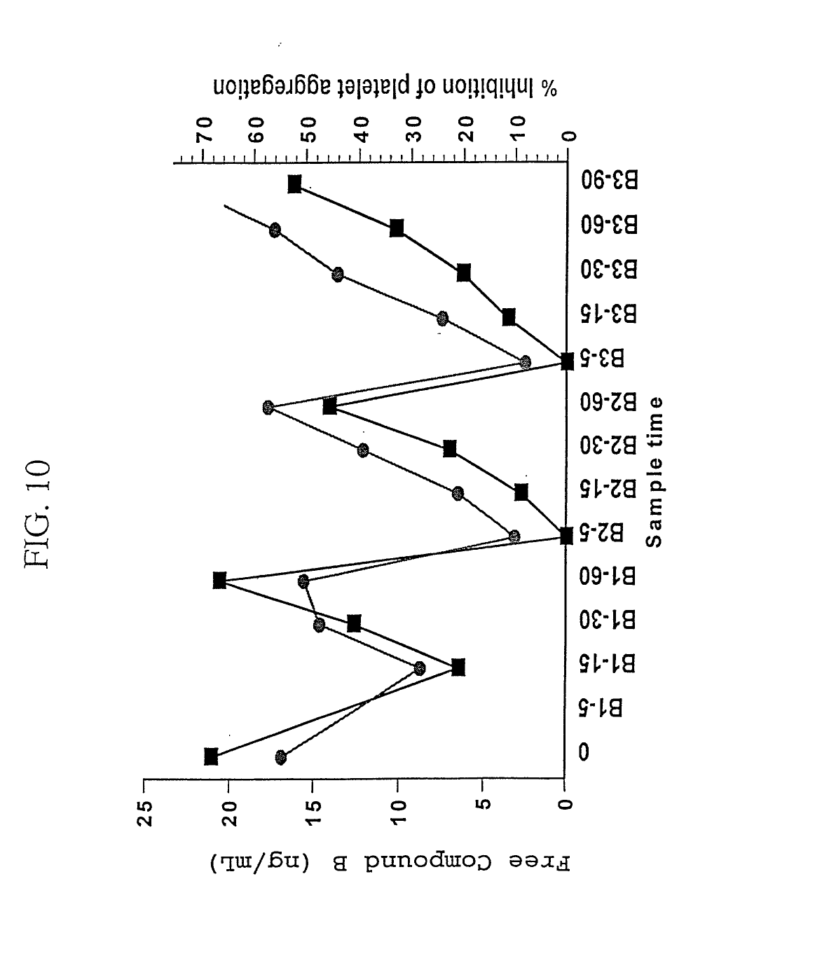

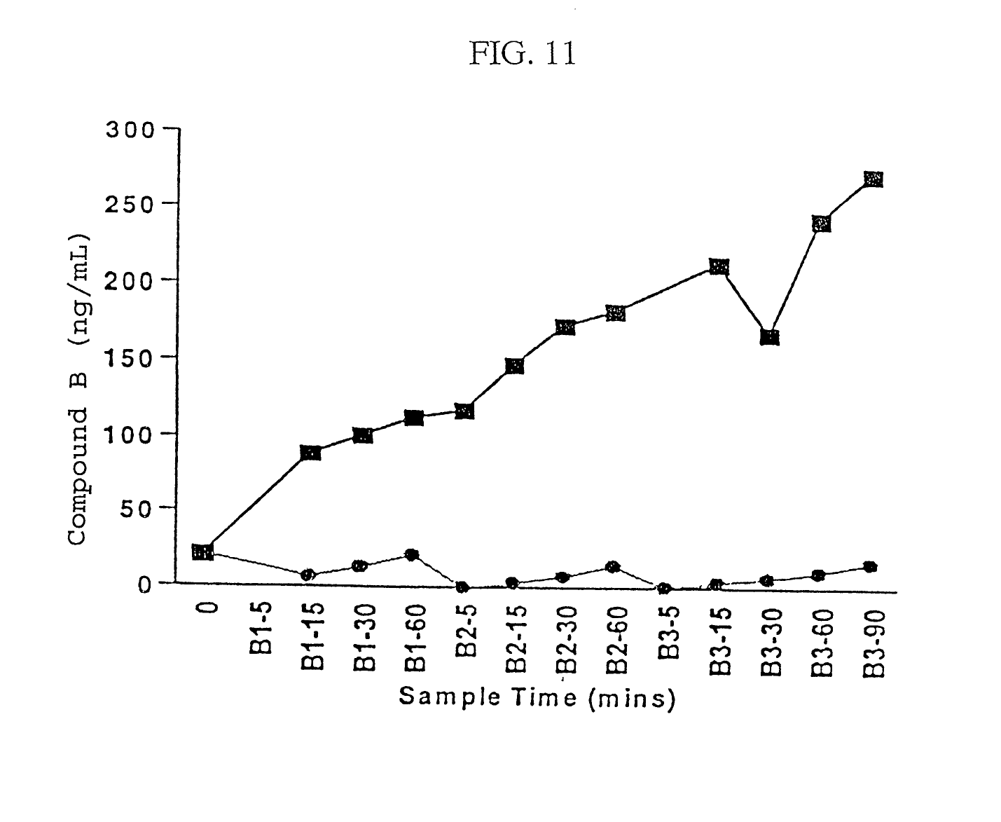

The bolus of Mab 9F7 reversed the platelet inhibition level and free plasma concentrations of compound B back to nearly baseline values 5 minutes after Mab 9F7 infusion. Upon cessation of the infusion of Mab 9F7, the platelet aggregation and free concentrations of compound B rose to the levels observed prior to the administration of the antibody (FIG. 10). Levels of total compound B (free plus antibody bound) increased throughout the three infusions of Mab 9F7 as the amount of antibody-bound drug increased with each bolus infusion (FIG. 11). As discussed below in detail, because Mab 9F7 was dosed based on plasma levels of compound B, rather than the total amount of systemically available compound B in the dog, it is not surprising that Mab 9F7 only transiently restored platelet function in this study.

### EXAMPLE 11

### Pre-Clinical Pharmokinetics (PK)

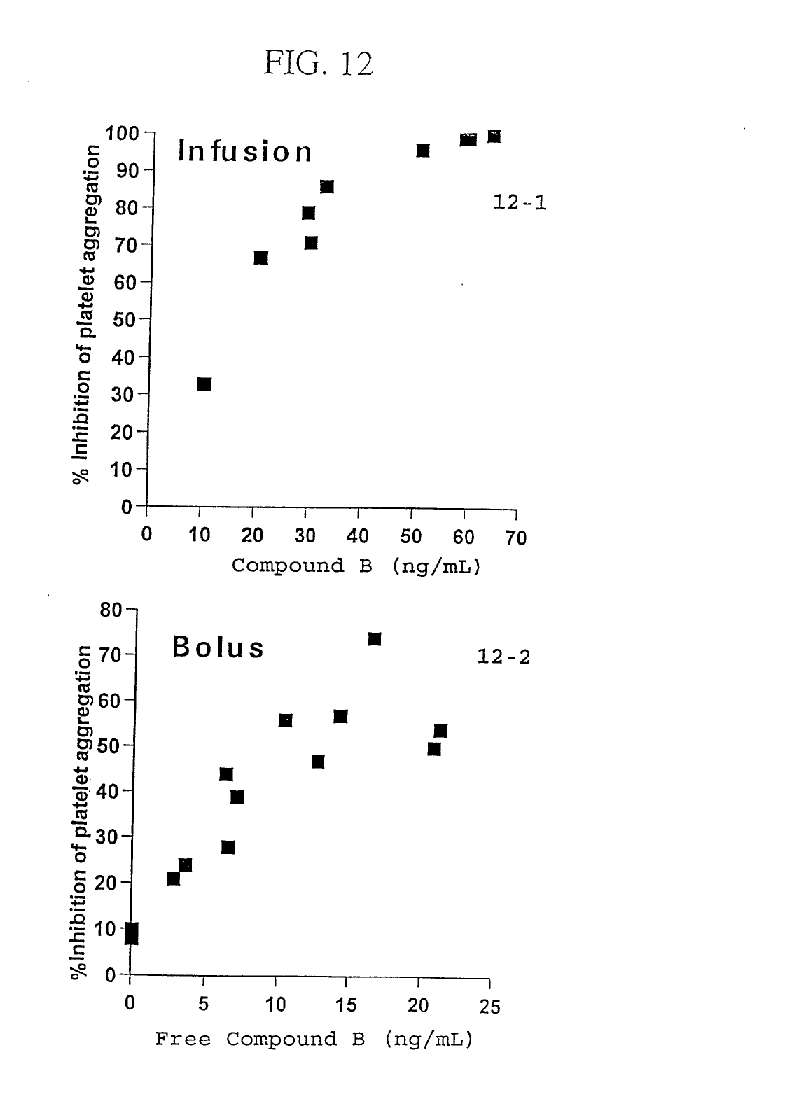

In the dog studies described above, the free concentrations of compound B correlated well with the degree of inhibition of platelet aggregation (FIG. 12).

The rebound in free concentrations of compound B in FIG. 10, and hence a reduction in the inhibition of platelet aggregation, is not unanticipated. The antibody binds available free compound B, and the compound B bound to the antibody is then replaced in the plasma as a result of distribution of free compound B from tissues into the plasma. In addition, the conversion of compound A to compound B is still occurring as well as absorption of compound A from the GI tract. Overall, these changes manifest themselves as a “rebound effect” as the amount of antibody administered is sufficient to only remove free compound B from the plasma but not sufficient to remove all compound B from the body (FIG. 10). Plasma concentrations of total compound B tended to be higher after administration of antibody relative to the concentrations before administration of the antibody. This reflects the summation of free compound B with the accumulation of antibody-bound compound B in the plasma.

The amount of antibody required to remove compound B from the plasma depends upon the timing of the administration relative to the dose of compound A and whether other treatments such as charcoal administration are being employed to block further absorption of compound A from the GI tract. This amount of antibody, like Mab 9F7, can be as high as 1 to 1.5 grams. Although this amount is large compared to the doses of Digibind (100-200 mg), it should be noted that Mab 9F7 is a full antibody and that Digibind is a Fab fragment. To avoid undue immunogenicity of the reversal therapy, an Fab fragment can be produced that lowers the mass of protein required approximately 3-fold. In addition, polyclonal antibodies are usually more potent than monoclonal antibodies due to significantly higher affinity for the drug ligand.

### EXAMPLE 12

### Measurement of GPIIb/IIIa Antagonist “Off-rate” from the receptor

1. Materials

Radioactive [³H]-compound B with a specific activity of 51.2 Ci/mMole was prepared at Chemsyn Science Laboratories, Lenexa, KS. Radiochemical purity was 99.76% as assessed by high performance liquid chromatography (HPLC) utilizing radiochemical detectors. Unlabelled compound B was prepared as described in U.S. Pat. No. 5,344,957. Compound B was assayed as a hydrated hydrochloride salt (Formula Weight :429 g/mole). Filters for the filter-binding assay were SSWP membrane with a pore size of 1.0 μm and were purchased from Millipore, Bedford, Mass. Scintillation fluid was Hionic-Fluor purchased from Packard Instrument Company (Meriden, Conn.). All other materials and reagents were of analytical grade.

2. Washed human platelet preparation

Sixty mL of human blood from the antecubital vein was collected into 1/10 volume of ACD (100 mM sodium citrate and 136 mM glucose, pH 6.5 with HCl). Platelet-rich plasma (PRP) was prepared by centrifugation of 30 mL of whole blood at 1000×g for 3 minutes without braking. PRP was removed from whole blood and placed in a 50 mL plastic centrifuge tube, PGE₁ (1.0×10⁻⁶ M) was added and the platelets were centrifuged for 10 minutes, 30 seconds at 900×g with no brake. The platelets were gently resuspended in modified Tyrodes buffer (137 mM NaCl, 2.6 mM KCl, 12 mM NaHCO₃, 5.5 mM glucose, 15 mM Hepes, 0.5 mg/mL bovine serum albumin, pH adjusted to 7.4 with NaOH), PGE₁ (1.0×10⁻⁶ M) was added to the wash buffer and the platelets recentrifuged, as above. The platelets were then gently resuspended in the modified Tyrodes buffer and a platelet count was determined by hemocytometer or Coulter Counter (Coulter Electronics, Hialeah, Fla. model S+IV). The platelet count was adjusted to 2.0×10⁸/mL with the modified Tyrodes buffer.

3. Filter Binding Assay

A filter-binding assay was used to demonstrate that the binding between platelets and GPIIb/IIIa receptor antagonist compounds is freely reversible and quite rapid. Washed platelets, prepared as described above, were incubated with 50 nM of [³H]-compound B for 20 min. A total volume of 15 mL was incubated in this manner in a 15 mL repipetor.

A 12-well Millipore filter manifold (Bedford, Mass.) was prepared with 1 μm filters just before the incubation period was completed. Wells 1 and 2 of the manifold were used for the zero time point and 1 mL of the incubation mixture was placed on each filter. At the zero time point, the incubated platelets were mixed with an excess of non-radioactive compound B by adding 1.8 mL of 1×10⁻³ M unlabeled compound B to the repeating pipettor as rapidly as possible. The platelets were then pipetted onto the filters at 1 second intervals until all the wells were filled. Because of the mixing step, the first time point was not sampled until at least 4 seconds.

The filter unit was maintained at 20 lbs of vacuum. After the samples were filtered and the filters dried, the filters were removed from the manifold and placed in scintillation vials. Samples from 5 and 10 minutes after introduction of the cold compound B were used to determine the non-specific binding of the filters. Twenty mL of scintillation cocktail were added to the scintillation vials and the vials were counted on a TmAnalytic 6881 Mark III scintillation counter for 2 minutes each.

4. Calculation of the “Off-rate”

Because the dissociation of the antagonist from the platelet surface can be described by a first-order rate equation, the results were analyzed by calculating the fraction of maximal counts bound at each time point after subtraction of the non-specific binding and plotting on a log scale vs. time. The slope of this plot yields the rate constant, k₋₁, divided by 2.303. The t_{½} can then be calculated as equal to 0.693/k₋₁.

### EXAMPLE 13

### Measurement of the GPIIb/IIIa Antagonist Plasma Half-life

### HPLC Analysis of Compound B in Dog Plasma

This method involves the extraction of compound B and an internal standard (compound F, shown hereinafter) from acidified dog plasma with a C₁₈ solid phase extraction column. Analysis is by reverse phase high performance liquid chromatography with fluorescence detection. Calibration standards were prepared in human heparinized plasma. Quality control pools were prepared in dog plasma and quantified using the human calibration curve. A linear weighted (1/concentration squared) least squares regression analysis was used to quantify samples. This method was validated with a minimum quantifiable level of 1.00 ng/mL. The sample was kept frozen at −70° C. prior to analysis and a 0.5 mL sample volume was required.

I. Preparation of Reagents

A. Mobile Phase

1. 0.1% Triethylamine in Phosphate Buffer (TEAP) pH=2.5

Added 2.00 g of potassium dihydrogen phosphate and 2.0 mL of triethylamine into a 2 liter container. Added 2 liters of Milli-Q™ water and mixed. While monitoring the pH, added phosphoric acid dropwise until the pH was 2.5. Stored at room temperature. Discarded after six months.

2. 0.1% TEAP (pH 2.5):methanol (75:25)

Added 1500 mL of 0.1% TEAP (pH 2.5) to 500 mL of methanol and mixed. Degassed by sparging with helium. Stored at room temperature. Discarded after 1 week.

B. Hydrochloric Acid (6 N)

Diluted 49.2 mL of hydrochloric acid (37%) to a 100 mL final volume with Milli-Q™ water. Stored at room temperature. Discarded after 1 year.

C. Hydrochloric Acid (0.025 N)

Diluted 4.2 mL of 6 N hydrochloric acid to a final volume of 1000 mL with Milli-Q™ water. Stored at room temperature. Discarded after three months.

D. Hydrochloric Acid (0.0025 N)

Diluted 10.0 mL of 0.025 N hydrochloric acid solution to a final volume of 100 mL with Milli-Q™ water. Stored at room temperature. Discarded after three months.

E. Acidified Water (pH 3.0)

While monitoring the pH of 1 liter of Milli-Q™ water, added phosphoric acid dropwise until the pH is 3.0. Stored at room temperature. Discarded after 1 month.

F. Formic Acid in Methanol (1%)

Diluted 1.0 mL of formic acid (88%) to a final volume of 100 mL with methanol. Stored at room temperature. Discarded after 1 month.

G. Water:Methanol (3:1 v/v)

Combined 125 mL of methanol with 375 mL of Milli-Q™ water. Stored at room temperature. Discarded after six months.

II. Preparation of Standards

A. Stock Solution A

compound B=1.00 mg/mL

Transferred 5.00 mg compound B to a 5.0 mL volumetric flask with approximately 2 ml of 50:50 methanol: Milli-Q. Added one drop of formic acid to clarify solution. Diluted to volume with methanol. Sonicated to dissolve. (Correct for salt form and purity.)

compound F=1.00 mg/mL

Transferred 5.00 mg compound E to a 5.0 mL volumetric flask and diluted to volume with methanol.

B. Working Internal Standard Solution C

compound F=100 ng/mL

Transferred 25 μL of Stock Solution B to a 250 mL volumetric flask. Diluted to volume with Milli-Q™ water.

C. Compound B Human Plasma Calibration Standards

Calibration standards were prepared in human heparinized plasma containing compound B at final concentrations of 1.00, 5.00, 10.0, 20.0, 50.0, 100 and 200 ng/mL. Plasma calibration standard 7 (200 ng/mL) was prepared first and the remaining calibration standards were dilutions of this standard, diluted to appropriate volumes with blank human plasma. After thorough mixing, freezed each calibration standard in daily portions at −70° C.

D. Compound B Dog Plasma Quality Control Pools

Quality control pools are prepared using heparinized dog plasma.

A second stock solution was made and quality control pools were prepared containing compound B at final concentrations of 2.50, 20.0, and 100 ng/mL. The highest concentration quality control pool (100 ng/mL) was prepared first and the other quality control pools were dilutions of this pool, diluted to appropriate volumes with blank dog plasma. After thorough mixing, each quality control pool was frozen in daily portions at −70° C.

III. Procedures

A. Blanks

A reagent blank, a human plasma blank, human plasma blank with internal standard, dog plasma blank, and a dog plasma blank with internal standard was prepared and extracted with each analysis run.

B. Calibration Standards, Samples and Quality Control Pools

Removed the samples to be analyzed from the freezer and thawed. Vortexed well. For the determination of unbound (“free”) plasma concentrations 0.500 mL of the dog plasma sample was placed in Amicon® Centrifree Unit (30 kD cut-off) and centrifuged for 1 hour at 2000 g av. and 4° C. The filtrate was then analyzed by the method given below. Transfered 0.500 mL of sample into a labeled 13×100 mm borosilicate culture tube. Recapped any remaining samples and returned them to the freezer immediately. Added 0.200 mL of Internal Standard Solution C to each tube. Added 0.500 mL of 0.0025 N hydrochloric acid to each tube. Vortexed. Activated a C₁₈ 100 mg SPEC for each sample as follows:

a. Filled the column with 2×1 mL of methanol and eluted slowly by vacuum.

b. Filled the column with 1 mL of water and elute slowly by vacuum.

c. Filled the column with 1 mL of acidified water (pH 3.0) and eluted slowly by vacuum.

d. Filled the column with 1 mL of acidified water (pH 3.0) and partially eluted slowly by vacuum without allowing the packing to dry.

Transferred each sample to an activated cartridge by decanting and eluted the plasma. Applied full vacuum for 5 seconds after all samples were passed through SPECs. Washed each SPEC column as follows:

a. Filled the column with 1 mL of acidified water (pH 3.0) and eluted by vacuum at moderate speed.

b. Repeated step 7a. Applied full vacuum for 10 seconds after all washes were passed through the SPECs.

c. Filled the column with 1 mL of Milli-Q™ water and eluted by vacuum at moderate speed.

d. Filled the column with 200 μL of methanol and eluted by vacuum at moderate speed. Applied full vacuum for 10 seconds after all washes were passed through the SPECs.

Filled each column with 1.0 mL of 1% formic acid in methanol and collected the eluate in 13×100 mm tubes. Applied full vacuum for 5 seconds after all the eluate was passed through the SPECs. Repeated. Evaporated under nitrogen and reconstituted with 150 μL of water:methanol, (3:1, v/v). Vortexed for 30 seconds. Transferred the samples to WISP inserts.

IV. Instrument Parameters

A. Chromatographic

Waters® WISP autosampler

Waters 501 pump

Guard Column: 12.5 mm×4.0 mm, 5 μm phenyl guard cartridge (replace after each run)

Analytical Column: 25 cm×4.6 mm, 5 μm Zorbax™ SB-phenyl column

Mobile Phase: 0.1% TEAP (pH 2.5):MeOH (75:25, v/v) Pumps A & B

Switching Valve Configuration:

Port 1: Guard Column Inlet

Port 2: Pump B

Port 3: Waste

Port 4: Guard Column Outlet

Port 5: Analytical Column

Port 6: Autosampler (Pump A)

Switching Program: Ports 1 and 6 on from 0 minute until Compound B and compound F elute from guard column. Return to starting position approximately 1.5 minutes prior to next injection.

Temperature: Ambient

Injection Volume: 25 μL

Flow Rate: 1 mL/min (Pump A and B)

B. Detector

Waters 470 Fluorescence Detector

Detector: Excitation: 275 nm Emission: 370 nm

Flow Cell: Standard

C. Integrator

Nelson Analytical System for IBM PC, Model 2600 Chromatography

VIII. Structures

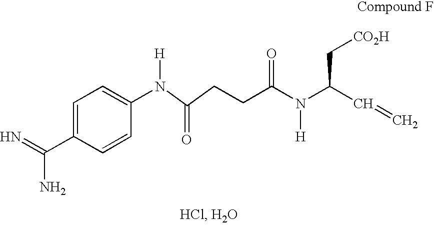

Analysis of data:

The half life of compound B was determined by fitting the plasma concentrations and determining the elimination rate constant kₑ. The half-life is calculated as t_{1/2}=0.693/kₑ

The foregoing description and the examples are intended as illustrative and are not to be taken as limiting. It is to be understood that no limitation with respect to the specific examples presented is intended or should be inferred. Still other variations within the spirit and scope of this invention are possible and will readily present themselves to those skilled in the art.

What is claimed is:

1. A process for restoring platelet aggregation in the blood of a mammalian host treated with a reversibly-bound GPIIb/IIIa receptor antagonist compound that exhibits a plasma half-life of about two hours to about thirty-six hours, and a GPIIb/IIIa receptor off-rate of about 0.7/seconds (t1/2˜1 second) to 0.012/seconds (t1/2˜60 seconds), or a pharmaceutically acceptable salt of said compound, that comprises the steps of:
(a) contacting the blood of said host with a therapeutically effective amount of antibody combining site-containing molecules that specifically bind to said GPIIb/IIIa receptor antagonist compound to form antibody-treated blood; and
(b) maintaining said antibody-treated blood for a period of time sufficient to restore platelet aggregation.

2. The process of claim 1 wherein said antibody combining site-containing molecules that specifically bind to said GPIIb/IIIa receptor antagonist are administered ex vivo.

3. The process of claim 1 wherein said antibody combining site-containing molecules that specifically bind to said GPIIb/IIIa receptor antagonist are administered in vivo.

4. The process of claim 3 wherein said antibody combining site-containing molecules that specifically bind to said GPIIb/IIIa receptor antagonist is parenterally administered.

5. The process of claim 1 wherein said mammalian host is selected from the group consisting of a dog, sheep, horse, cattle, goat, mouse, rat, ape, monkey, and a human.

6. The process of claim 5, wherein said mammalian host is a human.

7. The process of claim 1 wherein said antibody combining site-containing molecules that specifically bind to said GPIIb/IIIa receptor antagonist is an intact antibody.

8. The process of claim 1 wherein said antibody combining site-containing molecules that specifically bind to said GPIIb/IIIa receptor antagonist is free of immunoglobulin Fc portions.

9. The process of claim 1 wherein said antibody combining site containing molecules that specifically bind to said GPIIb/IIIa receptor antagonist is selected from the group consisting of a Fab, Fab′, F(ab′)₂, F(v), and a single chain antibody generated by phage display.

10. A process for restoring platelet aggregation in the blood of a mammalian host treated with a reversibly-bound GPIIb/IIIa receptor antagonist compound that exhibits a plasma half-life of about two hours to about thirty-six hours, and a GPIIb/IIIa receptor off-rate of about 0.7/seconds (t1/2˜1 second) to 0.012/seconds (t1/2˜60 seconds), or a pharmaceutically acceptable salt of said compound, that comprises the steps of:
(a) contacting the blood of said host in vivo with a therapeutically effective amount of antibody combining site-containing molecules that specifically bind to said GPIIb/IIIa receptor antagonist compound to form antibody-treated blood; and
(b) maintaining said antibody-treated blood for a period of time sufficient to restore platelet aggregation.

11. The process of claim 10 wherein said antibody combining site-containing molecules that specifically bind to said GPIIb/IIIa receptor antagonist are parenterally administered.

12. The process of claim 10 wherein said mammalian host is selected from the group consisting of a dog, sheep, horse, cattle, goat, mouse, rat, ape, monkey, and a human.

13. The process of claim 12 wherein the mammalian host is a human.

14. The process of claim 10 wherein said antibody combining site-containing molecules that specifically bind to said GPIIb/IIIa receptor antagonist are an intact antibodies.

15. The process of claim 10 wherein said antibody combining site-containing molecules that specifically bind to said GPIIb/IIIa receptor antagonist is free of immunoglobulin Fc portions.

16. The process of claim 10 wherein said antibody combining site containing molecules that specifically bind to said GPIIb/IIIa receptor antagonist is selected from the group consisting of a Fab, Fab′, F(ab′)₂, F(v), and a single chain antibody generated by phage display.

17. The process of claim 10 wherein said GPIIb/IIIa receptor antagonist is 3S-[[4-[[4-(aminoiminomethyl)-phenyl]amino]-1,4-dioxobutyl]amino]-4-pentynoic acid or (3-[[[[1-[4-(aminoiminomethyl)phenyl]-2-oxo-pyrrolidin-3S-yl]amino]carbonyl]amino]propanoic acid, or a pharmaceutically acceptable salt thereof.

18. The process of claim 10 wherein said antibody combining site-containing molecules that specifically bind to said GPIIb/IIIa receptor antagonist are a monoclonal antibodies.

19. The process of claim 17 wherein the monoclonal antibodies are antibody produced by a hybridoma designated ATCC HB-12081 or ATCC HB-12082.

20. The process of claim 10 wherein said antibody combining site-containing molecules that specifically bind to said GPIIb/IIIa receptor antagonist are polyclonal antibodies.

21. The process of claim 19 wherein said polyclonal antibodies are raised in a sheep or goat.

22. The process of claim 20 wherein said sheep or goat polyclonal antibodies are free of immunoglobulin Fc portions.
